(19) United States
(12) Patent Application Publication
(10) Pub. No.: US 2016/0130224 A1
(43) Pub. Date: May 12, 2016

(54) Novel Pyrrole Derivatives

(71) Applicant: UNIVERSITY OF LEICESTER, Leicester (GB)

(72) Inventors: Peter William ANDREW, Leicester (GB); Mafalda Pires DAMASO, Leicester (GB); Mark William DAVIES, Nottingham (GB); Fritz-Frieder FRICKEL, (US); Daniel HAMZA, Nottingham (GB); Simon Christopher HIRST, Nottingham (GB); Rana LONNEN, Leicester (GB)

(21) Appl. No.: 14/896,305

(22) PCT Filed: Jun. 4, 2014
(86) PCT No.: PCT/GB2014/051723

§ 371 Date: Dec. 4, 2015

(30) Foreign Application Priority Data
Jun. 4, 2013 (GB) 1309934.6

(51) Int. Cl. C07D 207/36; A61K 31/675; A61K 31/496; C07F 9/572; A61K 45/06

(52) U.S. Cl. CPC C07D 207/36; C07F 9/5722; A61K 45/06; A61K 31/496; A61K 31/675

(57) ABSTRACT

There are provided inter alia novel N-phenyl substituted pyrrole derivativesand theiruse in therapy, especially in the treatment of bacterial (e.g. pneumococcal) infections.

### FIELD OF THE INVENTION

The invention relates to compounds which are prodrugs of cytolysin inhibitors and their use in therapy, including in pharmaceutical combinations, especially in the treatment of bacterial, e.g. pneumococcal, infections.

### BACKGROUND OF THE INVENTION

Streptococcus pneumoniae (pneumococcus) is one of the most potent human pathogens, affecting over 10 million people worldwide, of all age groups, in particular young children, the elderly and the immunocompromised. It is a leading causative agent of serious, often fatal diseases, such as pneumonia, bacteraemia and meningitis. It is also responsible of other less serious, but nevertheless debilitating diseases such as otitis media and keratitis.

Even after decades of using antibiotics and steroids as adjunctive to antibiotics the mortality and morbidity from pneumococcal diseases remains very high in the developed world and alarmingly high in the developing world. Nearly 20% of hospitalised patients still die despite antibiotic killing of the pneumococcus, while many survivors of pneumococcal meningitis suffer severe neurological handicaps, including cognitive impairment, vision and hearing loss, hence imposing huge distress on patients and their families and a very significant cost to healthcare systems. Today, infection with pneumococcus remains a major global public health problem that is widely recognised by leaders in the field and by health organisations, including the WHO.

One of the leading factors for this consistently high mortality and morbidity that is not addressed by the current standard therapy, is the toxaemia resulting from the release of toxic pneumococcal products, the most important of which is the pneumococcal toxin pneumolysin. This toxin is a major player in pneumococcal virulence and is the primary direct and indirect cause of toxaemia.

Pneumolysin belongs to the family of cholesterol dependent cytolysins (CDCs), which bind to cholesterol containing membranes and generate large pores that have lethal and sub-lethal effects on the affected cells. In the bacterium, the toxin pneumolysin is cytoplasmic and is mainly released from the pneumococcus after its lysis. Consequently, under the effect of lytic antibiotics, a large bolus of toxin is released, compounding the toxaemia. Thus, even if treatment with antibiotics is successful in clearing the bacteria from the patients, the subsequent release of the toxin is detrimental and can be fatal or cause long-term handicaps.

This toxaemia constitutes a substantial unmet medical need that is internationally recognised. Currently, corticosteroids, principally dexamethasone, are used as an adjunctive to antibiotic therapy for pneumococcal meningitis. However, even when dexamethasone is used, significant mortality and morbidity are seen and the widespread use of dexamathasone is still debated due to its non-specific effect, limited clinical impact and in some cases its detrimental effect in increasing neuronal apoptosis in meningitis [Lancet (2002) 360 211-218]. Therefore, the present state of the art is not adequate for the efficient treatment of invasive pneumococcal diseases.

There is considerable evidence substantiating the validity of pneumolysin as a therapeutic target. In the laboratories of the inventors it has been demonstrated that, using a mouse pneumonia model, a mutated strain of S. pneumoniae (PLN-A) that does not produce pneumolysin is no longer lethal, causes substantially less bacteraemia and exhibits a significant reduction in the severity of pulmonary inflammation. Other evidence obtained in a rat meningitis model, has shown that infection with the pneumolysin-negative mutant was markedly less severe than with wild-type pneumococci, with no observed damage to the ciliated epithelium of the brain and no apoptosis of the cells surrounding the epithelium [J. Infect, (2007) 55 394-399]. In pneumococcal meningitis in guinea pigs, wild-type pneumococci induced severe cochlear damage and hearing loss, while infection with PLN-A left the organ of Corti intact [Infect. Immun. (1997) 65 4411-4418]. An ex vivo model using cultured ciliated brain epithelial cells, enabled recreation of the in vivo situation, where cells lining the brain ventricles are exposed to S. pneumoniae. Both intact and antibiotic-killed wild-type pneumococci induced damage to the epithelial cells in culture and significantly impaired ciliary beating; effects not seen with PLN-A [Infect. Immun. (2000) 68 1557-1562]. This damaging effect of antibiotic-lysed pneumococci on the cultured ependymal cells is clearly caused by the toxin pneumolysin released from the antibiotic-lysed bacteria, as this damage was abolished in the presence of anti-pneumolysin antibodies [Infect. Immun. (2004) 72 6694-6698]. This finding supports the strategy that antibiotic-induced toxaemia is prevented by combination with anti-pneumolysin agents.

Evidence for the significant involvement of pneumolysin in pneumococcal infections and the substantial improvement of the disease prognosis in the absence of pneumolsyin, has led to the conclusion that pneumolysin constitutes a potential therapeutic target to develop new treatments for pneumococcal diseases. Previous research has shown the ability of cholesterol to inhibit pneumolysin [Biochem. J. (1974) 140 95-98], however, this inhibition is merely due to the fact that cholesterol is a natural cellular receptor of pneumolysin that is required for the pore formation in the target cell membrane. The topical application of cholesterol on the cornea of rabbits demonstrated a positive therapeutic effect in pneumococcal keratitis [Invest. Ophtalmol. Vis. Sci. (2007) 48 2661-2666]. This indicates the involvement of pneumolysin in pneumococcal keratitis and the therapeutic benefit obtained following its inhibition. However, cholesterol is not considered as a therapeutic agent for the treatment of pneumococcal diseases and has not been clinically used in patients. Another pneumolysin inhibitor, Allicin, a component in garlic extract, has been previously found to inhibit the haemolytic activity of pneumolysin in vitro [Toxicon (2011) 57 540-545]. This compound is a cysteine inhibitor that irreversibly binds to the reactive thiol group of the toxin. Compounds exhibiting such a property are unfavourable as drug candidates because of their potential unspecific binding to other cysteine-containing proteins in the body.

There remains a need to provide inhibitors of cytolysins, such as pneumolysin, which are suitable for use in the treatment of bacterial infections.

International Patent Application PCT/GB2012/053022, published after the priority date of the present application and herein incorporated by reference in its entirety, discloses N-phenyl substituted pyrrole derivatives as cytolysin inhibitors, that specifically inhibit the direct toxic effect of pneumolysin and other cholesterol dependent cytolysins that are pivotal in the virulence of their respective hosts. These compounds have no structural similarity to Allicin and do not bind covalently to the reactive thiol groups of the toxins.

The present invention provides novel prodrugs of N-phenyl substituted pyrrole cytolysin inhibitors which prevent stimulation of host-derived toxic effects induced by pneumolysin and, it may be assumed, other cholesterol dependent cytolysins. The compounds appear also to demonstrate good aqueous solubility and good chemical stability in aqueous solution. Thus the compounds may be used as single agents or as an adjunct to antibiotics, to prevent or attenuate pneumolysin-induced toxicity and its anti-host effects seen during infections caused e.g. by S. pneumoniae.

### SUMMARY OF THE INVENTION

According to the invention, there is provided a compound selected from:

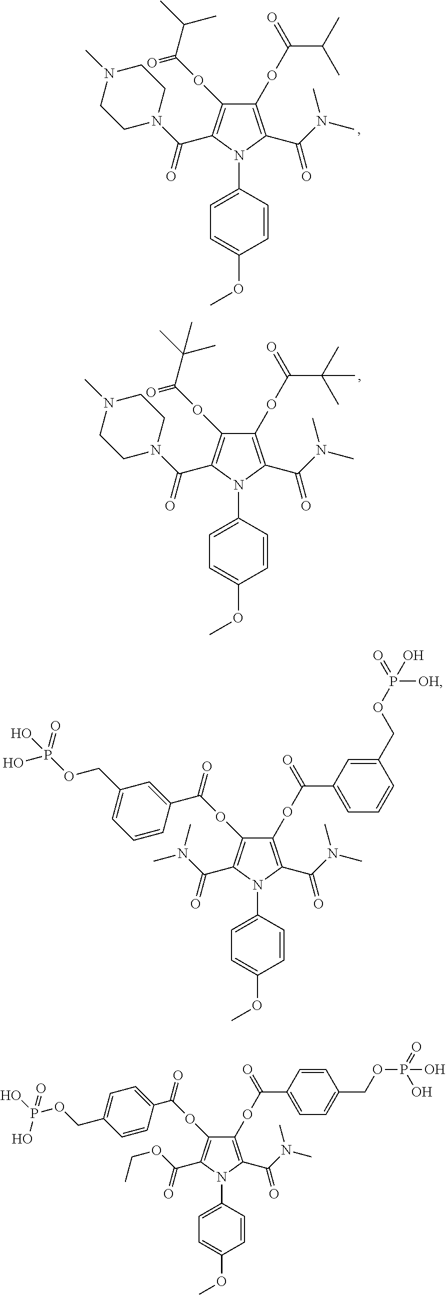

and pharmaceutically acceptable salts and solvates thereof.

In a further aspect, the present invention provides a compound as defined above (hereinafter referred to as a compound of the invention) for use as a medicament.

### BRIEF DESCRIPTION OF THE FIGS.

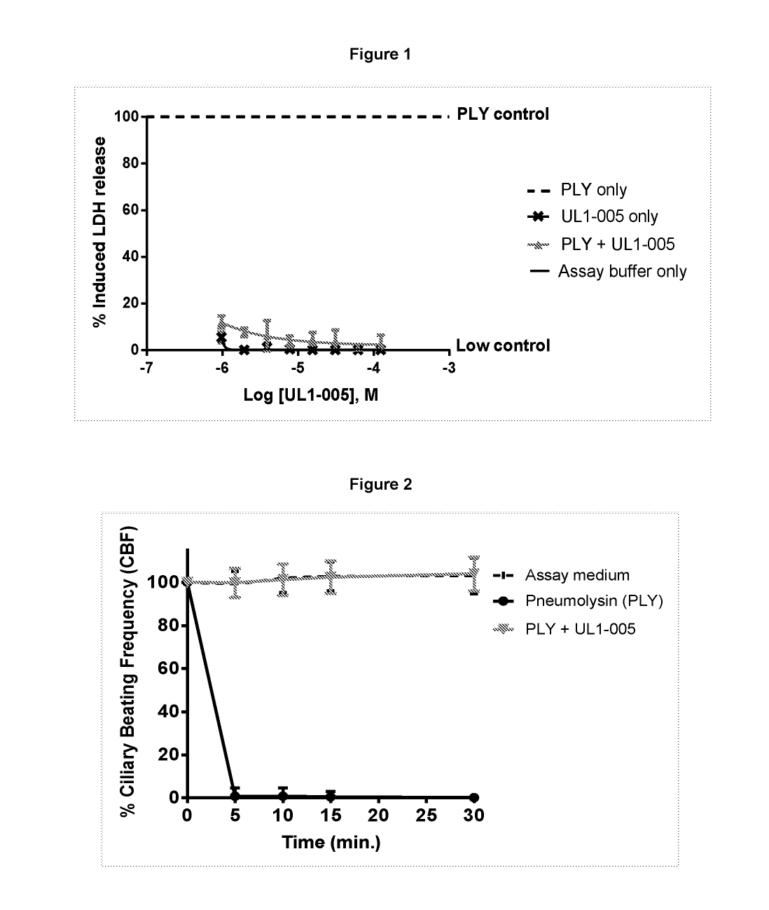

FIG. 1 shows the in vitro inhibition of pneumolysin-induced LDH release by the compound UL1-005 using A549 human lung epithelial cells.

FIG. 2 shows the effect of the compound UL1-005 in inhibiting pneumolysin from damaging the ciliary function of ependymal cells in an ex vivo meningitis efficacy assay.

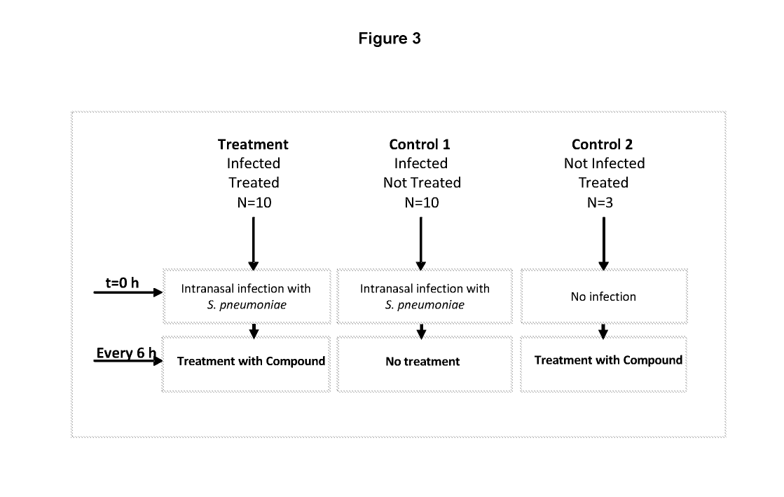

FIG. 3 shows the experimental design for an in vivo mouse pneumonia model efficacy assay using a compound of the invention.

### DETAILED DESCRIPTION OF THE INVENTION

The compound of the invention are prodrug derivatives of the corresponding 3,4-dihydroxy pyrrole derivatives. A compound of the invention will break down after administration to a subject to form the active 3,4-dihydroxy compound (sometimes referred to herein as “parent active compound”) in vivo.

Examples of salts of the compounds of the invention include all pharmaceutically acceptable salts prepared from pharmaceutically acceptable non-toxic bases or acids. Salts derived from bases include, for example, potassium and sodium salts and the like. Salts derived from acids, include those derived from inorganic and organic acids such as, for example, hydrochloric, methanesulfonic, sulfuric and p-toluenesulfonic acid and the like.

Examples of solvates of the compounds of the invention include hydrates.

The invention includes solvates (including hydrates) of salts.

Particular examples of the compounds of the invention which may be mentioned include:

2-(Dimethylcarbamoyl)-1-(4-methoxyphenyl)-5-(4-methylpiperazine-1-carbonyl)-1H-pyrrole-3,4-diyl bis(2-methylpropanoate),

2-(Dimethylcarbamoyl)-1-(4-methoxyphenyl)-5-(4-methylpiperazine-1-carbonyl)-1H-pyrrole-3,4-diyl bis(2-methylpropanoate) hydrochloride,

2-(Dimethylcarbamoyl)-1-(4-methoxyphenyl)-5-(4-methylpiperazine-1-carbonyl)-1H-pyrrole-3,4-diyl bis(2,2-dimethylpropanoate),

2-(Dimethylcarbamoyl)-1-(4-methoxyphenyl)-5-(4-methylpiperazine-1-carbonyl)-1H-pyrrole-3,4-diylbis(2,2-dimethylpropanoate)hydrochloride,

2,5-Bis(dimethylcarbamoyl)-1-(4-methoxyphenyl)-1H-pyrrole-3,4-diyl bis(3-((phosphonooxy)methyl)benzoate),

Sodium ((((2,5-bis(dimethylcarbamoyl)-1-(4-methoxyphenyl)-1H-pyrrole-3,4-diyl)bis(oxy))bis(carbonyl))bis(3,1-phenylene))bis(methylene) bis(hydrogenphosphate),

2-(Dimethylcarbamoyl)-5-(ethoxycarbonyl)-1-(4-methoxyphenyl)-1H-pyrrole-3,4-diylbis(4-((phosphonooxy)methyl)benzoate), and

Sodium ((((2-(dimethylcarbamoyl)-5-(ethoxycarbonyl)-1-(4-methoxyphenyl)-1H-pyrrole-3,4-diyl)bis(oxy))bis(carbonyl))bis(4,1-phenylene))bis(methylene)bis(hydrogenphosphate).

The invention also extends to all polymorphic forms of the compounds of the invention.

The invention also extends to isotopically-labelled compounds of the invention in which one or more atoms are replaced by an atom having an atomic mass or mass number different from the atomic mass or mass number most commonly found in nature. Examples of isotopes that can be incorporated into compounds of the invention include isotopes of hydrogen, carbon, nitrogen, and phosphorus, such as ²H, ³H, ¹¹C, ¹⁴C, ¹⁵N, ³²P and ³³P. Isotopically labelled compounds of the invention may be prepared by carrying out the synthetic methods described below and substituting an isotopically labelled reagent or intermediate for a non-isotopically labelled reagent or intermediate.

The compounds of the invention may be prepared as described in the Examples.

Thus according to a further aspect of the invention there is provided a process for the production of the compounds of the invention which comprises reacting a compound of formula (I):

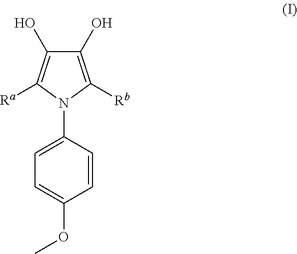

wherein R^{a} and R^{b} correspond to the 2- and 5-position substituents in the compounds of the invention, with:

a) 3-((phosphonooxy)methyl) benzoic acid), or a protected derivative thereof e.g. a di-tert-butyl protected derivative thereof, followed if required by deprotection; or

b) a compound of formula LG-C(O)—R^{c}, where LG is a leaving group e.g. chloro and R^{c} is —C(CH₃)₃ or —CH(CH₃)₂;

and optionally forming a salt or solvate thereof.

Any novel intermediates may be of use in the synthesis of the compounds of the invention and are therefore also included within the scope of the invention.

Protecting groups may be required to protect chemically sensitive groups during the synthesis of the compound of the invention, to ensure that the process is efficient. Thus if desired or necessary, intermediate compounds may be protected by the use of conventional protecting groups. Protecting groups and means for their removal are described in “Protective Groups in Organic Synthesis”, by Theodora W. Greene and Peter G. M. Wuts, published by John Wiley & Sons Inc; 4^{th} Rev Ed., 2006, ISBN-10: 0471697540.

As indicated above the compounds of the invention are useful for treatment of bacterial infections caused by bacteria producing pore-forming toxins, such as cholesterol dependent cytolysins.

In particular the compounds of the invention are useful for the treatment of toxaemia associated with bacterial infections.

For such use the compounds of the invention will generally be administered in the form of a pharmaceutical composition.

Further, the present invention provides a pharmaceutical composition comprising a compound of the invention optionally in combination with one or more pharmaceutically acceptable diluents or carriers.

Diluents and carriers may include those suitable for parenteral, oral, topical, mucosal and rectal administration.

As mentioned above, such compositions may be prepared e.g. for parenteral, subcutaneous, intramuscular, intravenous, intra-articular or peri-articular administration, particularly in the form of liquid solutions or suspensions; for oral administration, particularly in the form of tablets or capsules; for topical e.g. intravitreal, pulmonary or intranasal administration, particularly in the form of eye drops, powders, nasal drops or aerosols and transdermal administration; for mucosal administration e.g. to buccal, sublingual or vaginal mucosa, and for rectal administration e.g. in the form of a suppository.

The compositions may conveniently be administered in unit dosage form and may be prepared by any of the methods well-known in the pharmaceutical art, for example as described in Remington's Pharmaceutical Sciences, 17th ed., Mack Publishing Company, Easton, Pa., (1985). Formulations for parenteral administration may contain as excipients sterile water or saline, alkylene glycols such as propylene glycol, polyalkylene glycols such as polyethylene glycol, oils of vegetable origin, hydrogenated naphthalenes and the like. Formulations for parenteral administration may be provided in solid form, such as a lyophilised composition, the lyophilised composition may be re-constituted, preferably just before administration. Re-constitution may involve dissolving the lyophilised composition in water or some other pharmaceutically acceptable solvent, for example physiological saline, an aqueous solution of a pharmaceutically acceptable alcohol, e.g. ethanol, propylene glycol, a polyethylene glycol, e.g. polyethylene glycol 300, and the like, or some other sterile injectable.

Formulations for nasal administration may be solid and may contain excipients, for example, lactose or dextran, or may be aqueous or oily solutions for use in the form of nasal drops or metered spray. For buccal administration typical excipients include sugars, calcium stearate, magnesium stearate, pregelatinated starch, and the like.

Compositions suitable for oral administration may comprise one or more physiologically compatible carriers and/or excipients and may be in solid or liquid form. Tablets and capsules may be prepared with binding agents, for example, syrup, acacia, gelatin, sorbitol, tragacanth, or poly-vinylpyrollidone; fillers, such as lactose, sucrose, corn starch, calcium phosphate, sorbitol, or glycine; lubricants, such as magnesium stearate, talc, polyethylene glycol, or silica; and surfactants, such as sodium lauryl sulfate. Liquid compositions may contain conventional additives such as suspending agents, for example sorbitol syrup, methyl cellulose, sugar syrup, gelatin, carboxymethyl-cellulose, or edible fats; emulsifying agents such as lecithin, or acacia; vegetable oils such as almond oil, coconut oil, cod liver oil, or peanut oil; preservatives such as butylated hydroxyanisole (BHA) and butylated hydroxytoluene (BHT). Liquid compositions may be encapsulated in, for example, gelatin to provide a unit dosage form.

Solid oral dosage forms include tablets, two-piece hard shell capsules and soft elastic gelatin (SEG) capsules.

A dry shell formulation typically comprises of about 40% to 60% concentration of gelatin, about a 20% to 30% concentration of plasticizer (such as glycerin, sorbitol or propylene glycol) and about a 30% to 40% concentration of water. Other materials such as preservatives, dyes, opacifiers and flavours also may be present. The liquid fill material comprises a solid drug that has been dissolved, solubilized or dispersed (with suspending agents such as beeswax, hydrogenated castor oil or polyethylene glycol 4000) or a liquid drug in vehicles or combinations of vehicles such as mineral oil, vegetable oils, triglycerides, glycols, polyols and surface-active agents.

Pharmaceutical compositions of the invention may optionally include one or more anti-oxidants (e.g. ascorbic acid or metabisulfate and salts thereof).

Particular pharmaceutical compositions according to the invention which may be mentioned include the following:
A pharmaceutical composition for parenteral, e.g. intravenous, administration.
A pharmaceutical composition for oral administration.
A pharmaceutical composition for parenteral, e.g. intravenous, or oral administration in unit dose form.
A pharmaceutical composition for parenteral, e.g. intravenous, administration in solid form for reconstitution with a liquid prior to administration.
A pharmaceutical composition for parenteral, e.g. intravenous, administration in liquid form e.g. a solution.

The compounds of the invention are inhibitors of the cholesterol-dependent cytolysin, pneumolysin, produced by the bacterium Streptococcus pneumoniae. It also inhibits Streptolysin O (SLO) produced by Group A Streptococci and Perfringolysin O (PFO) produced by Clostridium perfringens. They are also expected to inhibit other members of the closely related cholesterol-dependent cytolysins, examples of which include, but are not limited to, Listeriolysin O (LLO) produced by Listeria monocytogenes, Anthrolysin O (ALO) produced by Bacillus anthracis and Suilysin (SLY) produced by Streptococcus suis.

The compounds of the invention are useful for the treatment of bacterial infections, e.g. pneumococcal infections including the associated toxaemia where the pneumolysin toxin has been demonstrated to play a pivotal role in the diseases produced. Such diseases include, but are not limited to, pneumococcal pneumonia, pneumococcal meningitis, pneumococcal septicaemia/bacteraemia, pneumococcal keratitis and pneumococcal otitis media. The compounds of the invention are also useful for the treatment of pneumococcal infections associated with other conditions. Such conditions include (without limitation) cystic fibrosis and chronic obstructive pulmonary disease (COPD). For example, S pneumoniae has been isolated from patients with COPD and is believed to be an exacerbatory factor in this disease.

The compounds of the invention are useful for the treatment of infections caused by group A Streptococci (GAS), including but not limited to, invasive group A Streptococcal diseases, where the toxin Streptolysin O (SLO) has been demonstrated to play a crucial role in the pathogenesis of systemic GAS diseases.

The compounds of the invention are useful for the treatment of infections caused by Clostridium perfringens including, but not limited to, gas gangrene, characterized by myonecrosis, septic shock and death, where the toxin Perfringolysin O has been demonstrated to be a major virulence factor in the pathogenesis of this disease.

The compounds of the invention are useful for the treatment of infections caused by Bacillus anthracis, where the cholesterol dependent cytolysin Anthrolysin O (ALO) plays an essential role in gastrointestinal (GI) anthrax, and contributes to the pathogenesis of inhalational anthrax.

The compounds of the invention are useful for the treatment of other diseases caused by Gram positive bacteria, producing cholesterol-dependent cytolysins, examples of which include, but are not limited to:

Porcine meningitis, septicaemia/bacteraemia and septic shock caused by Streptococcus suis which produces a cholesterol dependent cytolysin, Suilysin, involved in the pathogenesis of diseases by S. suis.

Encephalitis, enteritis, meningitis, septicaemia/bacteraemia and pneumonia caused by Listeria monocytogenes where the cholesterol dependent cytolysin, listeriolosin O (LLO), plays an important role in the pathogensis of the above diseases.

The compounds of the invention may well also be useful for the inhibition of other bacterial pore-forming toxins, such as the RTX family of toxins, which are essential in the virulence of their host. Examples include, but are not limited to, pneumonia and septicaemia/bacteraemia caused by Staphylococcus aureus, which produces the pore-forming toxin staphylococcal α-hemolysis and peritonitis caused by pathogenic Escherichia coli which produces the pore forming toxin α-hemolysin.

Thus the invention provides:
A compound of the invention for use in the treatment of bacterial infections caused by bacteria producing pore-forming toxins, wherein the bacterial infection is caused by Streptococcus spp. (e.g. Streptococcus pneumoniae, Group A Streptococci or Streptococcus suis), Clostridium spp. (e.g. Clostridium perfringens), Listeria spp. (e.g. Listeria monocytogenes) or Bacillus spp. (e.g. Bacillus anthracis);
A compound of the invention for the treatment of bacterial infection which is caused by Streptococcus pneumonia;
A compound of the invention for use in the treatment of pneumococcal pneumonia, pneumococcal meningitis, pneumococcal septicaemia/bacteraemia, pneumococcal keratitis or pneumococcal otitis media; and
A compound of the invention for the treatment of conditions selected from gas gangrene, gastrointestinal anthrax, inhalational anthrax, porcine meningitis, encephalitis, septicaemia/bacteraemia and pneumonia which are caused by bacteria other than pneumococcus.

The compounds of the invention may be used to treat either humans or animals, such as domestic animals or livestock, e.g. pigs, cows, sheep, horses etc, and references to pharmaceutical compositions should be interpreted to cover compositions suitable for either human or animal use.

Thus, in a further aspect, the present invention provides a compound of the invention for use in the treatment of the above mentioned conditions.

In a further aspect, the present invention provides a compound of the invention for the manufacture of a medicament for the treatment of the above mentioned conditions.

In a further aspect, the present invention provides a method of treatment of the above mentioned conditions which comprises administering to a subject in need thereof an effective amount of a compound of the invention or a pharmaceutical composition thereof.

The word “treatment” is intended to embrace prophylaxis as well as therapeutic treatment.

The compounds of the invention may be used either alone or in combination with further therapeutically active ingredients. Thus compounds of the invention may be administered in combination, simultaneously, sequentially or separately, with further therapeutically active ingredients either together in the same formulation or in separate formulations and either via the same route or via a different route of administration. The compounds of the invention may thus be administered in combination with one or more other active ingredients suitable for treating the above mentioned conditions. For example, possible combinations for treatment include combinations with antimicrobial agents, e.g. antibiotic agents, including natural, synthetic and semisynthetic antimicrobial agents. Examples of antibiotic agents include β-lactams including, but not limited to, penicillin, benzylpenicillin, amoxicillin and all generations thereof; β-lactams in combination with β-lactamase inhibitors including, but not limited to, clavulanic acid and sulbactam; cephalosporins including, but not limited to, cefuroxime, cefotaxime and ceftriaxone; fluoroquinolones including, but not limited to, levofloxacin and moxifloxacin; tetracyclines including, but not limited to, doxycycline; macrolides including, but not limited to, erythromycin and clarithromycin; lipopeptide antibiotics including, but not limited to, daptomycin; aminoglycosides including, but not limited to, kanamycin and gentamicin; glycopeptide antibiotics, including but not limited to, vancomycin; lincosamides including, but not limited to, clindamycin and lincomycin; rifamycins including, but not limited to, rifampicin; and chloramphenicol.

Further combinations include combinations with immunomodulatory agents, such as anti-inflammatory agents.

Immunomodulatory agents can include for example, agents which act on the immune system, directly or indirectly, by stimulating or suppressing a cellular activity of a cell in the immune system, for example, T-cells, B-cells, macrophages, or antigen presenting cells, or by acting upon components outside the immune system which, in turn, stimulate, suppress, or modulate the immune system, for example, hormones, receptor agonists or antagonists and neurotransmitters, other immunomodulatory agents can include immunosuppressants or immunostimulants. Anti-inflammatory agents include, for example, agents which treat inflammatory responses, tissue reaction to injury, agents which treat the immune, vascular or lymphatic systems or combinations thereof. Examples of anti-inflammatory and immunomodulatory agents include, but are not limited to, interferon derivatives such as betaseron, β-interferon, prostane derivatives such as iloprost and cicaprost, corticosteroids such as prednisolone, methylprednisolone, dexamethasone and fluticasone, COX2 inhibitors, immunsuppressive agents such as cyclosporine A, FK-506, methoxsalene, thalidomide, sulfasalazine, azathioprine and methotrexate, lipoxygenase inhibitors, leukotriene antagonists, peptide derivatives such as ACTH and analogs, soluble TNF (tumor necrosis factor)-receptors, TNF-antibodies, soluble receptors of interleukines, other cytokines and T-cell-proteins, antibodies against receptors of interleukins, other cytokines and T-cell-proteins. Further anti-inflammatory agents include non-steroidal anti-inflammatory drugs (NSAID's). Examples of NSAID's include sodium cromoglycate, nedocromil sodium, phosphodiesterase (PDE) inhibitors e.g. theophylline, PDE4 inhibitors or mixed PDE3/PDE4 inhibitors, leukotriene antagonists, inhibitors of leukotriene synthesis such as montelukast, iNOS inhibitors, tryptase and elastase inhibitors, beta-2 integrin antagonists and adenosine receptor agonists or antagonists such as adenosine 2a agonists, cytokine antagonists e.g. chemokine antagonists, such as CCR3 antagonists, or inhibitors of cytokine synthesis, and 5-lipoxygenase inhibitors.

Thus an aspect of the invention provides a compound of the invention in combination with one or more further active ingredients, for example one or more of the active ingredients described above.

Another aspect of the invention provides a pharmaceutical composition comprising a compound of the invention optionally in combination with one or more pharmaceutically acceptable adjuvants, diluents or carriers and comprising one or more other therapeutically active ingredients.

Similarly, another aspect of the invention provides a combination product comprising:

(A) a compound of the invention; and

(B) another therapeutic agent,

wherein each of components (A) and (B) is formulated in admixture with a pharmaceutically-acceptable adjuvant, diluent or carrier.

In this aspect of the invention, the combination product may be either a single (combination) pharmaceutical formulation or a kit-of-parts.

Thus, this aspect of the invention encompasses a pharmaceutical formulation including a compound of the invention and another therapeutic agent, in admixture with a pharmaceutically acceptable adjuvant, diluent or carrier (which formulation is hereinafter referred to as a “combined preparation”).

It also encompasses a kit of parts comprising components:
a pharmaceutical formulation including a compound of the invention in admixture with a pharmaceutically acceptable adjuvant, diluent or carrier; and
(ii) a pharmaceutical formulation including another therapeutic agent, in admixture with a pharmaceutically-acceptable adjuvant, diluent or carrier;

which components (i) and (ii) are each provided in a form that is suitable for administration in conjunction with the other.

Component (i) of the kit of parts is thus component (A) above in admixture with a pharmaceutically acceptable adjuvant, diluent or carrier. Similarly, component (ii) is component (B) above in admixture with a pharmaceutically acceptable adjuvant, diluent or carrier.

The other therapeutic agent (i.e. component (B) above) may be, for example, any of the agents e.g. antimicrobial or immunomodulatory agents mentioned above.

The combination product (either a combined preparation or kit-of-parts) of this aspect of the invention may be used in the treatment or prevention of any of the conditions mentioned above.

The compounds of the invention may also be provided for use, e.g. with instructions for use, in combination with one or more further active ingredients.

Thus a further aspect of the invention provides a compound of formula (I) for use in combination with one or more further active ingredients, for example one or more of the active ingredients described above.

The compounds of the invention for use in this aspect of the invention may be used in the treatment or prevention of any of the conditions mentioned above.

The invention will now be described by reference to the following examples which are for illustrative purposes and are not to be construed as a limitation of the scope of the present invention.

### EXAMPLES

Abbreviations

AcOH glacial acetic acid

aq. aqueous

Bn benzyl

br broad

Boc tert-butoxycarbonyl

COPD chronic obstructive pulmonary disease

d doublet

DCM dichloromethane

DIPEA N,N-diisopropylethylamine

DMAP 4-dimethylaminopyridine

DMF N,N-dimethylformamide

DMSO dimethylsulfoxide

EDC 1-ethyl-3-(3-dimethylaminopropyl)carbodiimide

EtOAc ethyl acetate

h hour(s)

HATU N,N,N′,N′-tetramethyl-O-(7-azabenzotriazol-1-yl)uronium PF₆

HPLC high performance liquid chromatography

m multiplet

MeCN acetonitrile

MeOH methanol

min minute(s)

NMR nuclear magnetic resonance

PBS phosphate buffered saline

quin. quintet

RT room temperature

s singlet

sat. saturated

SAX solid supported strong cation exchange resin

sept. septet

sext. sextet

t triplet

TFAA trifluoroacetic acid anhydride

THF tetrahydrofuran

UV ultra violet

General Procedures

All starting materials and solvents were obtained from commercial sources or prepared according to literature conditions.

Hydrogenations were performed either on a Thales H-cube flow reactor or with a suspension of the catalyst under a balloon of hydrogen.

Column chromatography was performed on pre-packed silica (230-400 mesh, 40-63 μM) cartridges.

PBS solutions for solubility and stability studies were prepared by dissolving 1 Oxoid™ tablet (obtained from Thermo Scientific) in deionised water (100 mL).

Stability studies were carried out by dissolving 1-2 mg of compound in DMSO (1 mL) followed by addition of 0.4 mL of the resulting solution to stirred PBS solution (9.6 mL) at 37.5° C. A sample (ca. 0.5 mL) was immediately taken for HPLC analysis. Further samples were then taken for analysis at various timepoints thereafter. Half-lives were determined from the decrease in concentration of compound with respect to time.

Analytical Methods

Analytical HPLC was carried out using an Agilent Zorbax Extend C18, Rapid Resolution HT 1.8 μm column eluting with a 5-95% gradient of either 0.1% formic acid in MeCN in 0.1% aqueous formic acid or a 5-95% gradient of MeCN in 50 mM aqueous ammonium acetate. Alternatively, a Waters Xselect CSH C18 3.5 μm eluting with a 5-95% gradient of 0.1% formic acid in MeCN in 0.1% aqueous formic acid. UV spectra of the eluted peaks were measured using either a diode array or variable wavelength detector on an Agilent 1100 system.

Analytical LCMS was carried out using an Agilent Zorbax Extend C18, Rapid Resolution HT 1.8 μm column eluting with a 5-95% gradient of either 0.1% formic acid in MeCN in 0.1% aqueous formic acid or a 5-95% gradient of MeCN in 50 mM aqueous ammonium acetate. Alternatively, a Waters Xselect CSH C18 3.5 μm eluting with a 5-95% gradient of 0.1% formic acid in MeCN in 0.1% aqueous formic acid. UV and mass spectra of the eluted peaks were measured using a variable wavelength detector on either an Agilent 1100 with or an Agilent Infinity 1260 LC with 6120 quadrupole mass spectrometer with positive and negative ion electrospray.

Preparative HPLC was carried out using an Agilent Prep-C18 5 pm Preparative Cartridge using either a gradient of 0.1% formic acid in MeCN in 0.1% aqueous formic acid or a gradient of MeCN in 10 mM Ammonium Bicarbonate, Alternatively, a Waters Xselect CSH C18 5 μm column using a gradient 0.1% MeCN in 0.1% aqueous formic acid. Fractions were collected following detection by UV at 254 nm.

¹H NMR Spectroscopy:

NMR spectra were recorded using a Bruker Avance III 400 MHz instrument, using either residual non-deuterated solvent or tetra-methylsilane as reference.

Chemical Synthesis:

The compounds of the invention, corresponding parent compounds and comparator compound were prepared using the following general methods.

### Example A1

### 3,4-Dihydroxy-1-(4-methoxyphenyl)-N²,N²,N⁵,N⁵-tetramethyl-1H-pyrrole-2,5-dicarboxamide (UL1-005)

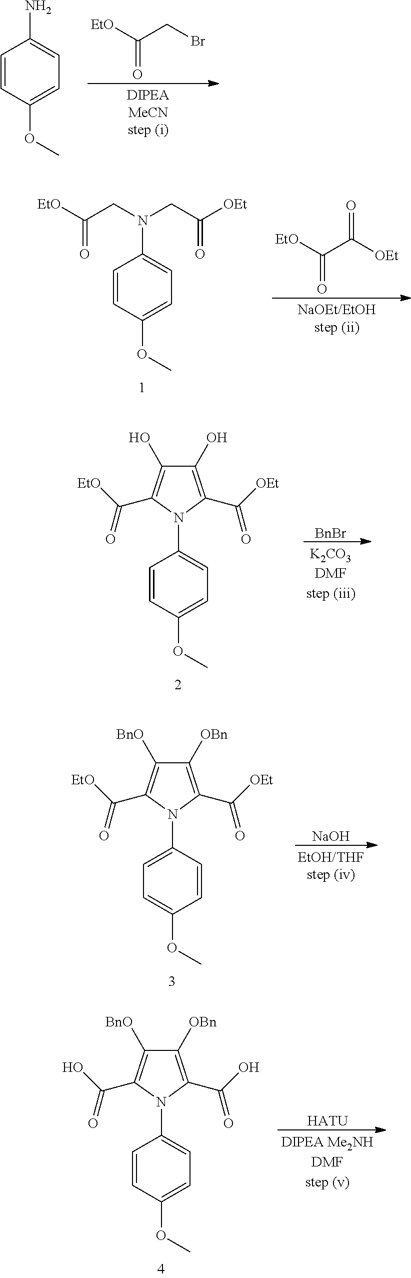

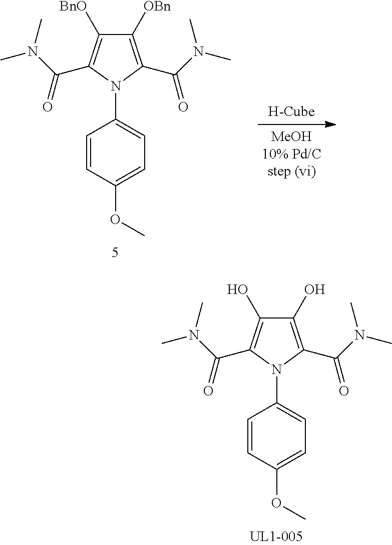

### Step (i): Diethyl 2,2′-((4-methoxyphenyl)azanediyl)diacetate (1)

Ethyl 2-bromoacetate (146 mL, 1.30 mol) was added dropwise to a stirred solution of 4-methoxyaniline (75.0 g, 0.610 mol) and DIPEA (265 mL, 1.50 mol) in MeCN (300 mL). The reaction mixture was stirred at 60° C. for 16 h and then partitioned between 2 M HCl_{(aq.)} (500 mL), and EtOAc (300 mL), the aqueous phase was extracted with EtOAc (300 mL) and the combined organics were washed succesively with 2M HCl_{(aq.)} (2×300 mL), water (500 mL), and brine (500 mL), dried (MgSO₄), filtered and solvents removed in vacuo to give diethyl 2,2′-((4-methoxyphenyl)azanediyl)diacetate (1) (180 g, 100%) as a purple oil: m/z 296 (M+H)⁺ (ES⁺). ¹H NMR (400 MHz, CDCl₃) δ 6.82-6.78 (m, 2H), 6.64-6.59 (m, 2H), 4.19 (q, J=7.1 Hz, 4H), 4.10 (s, 4H), 3.74 (s, 3H), 1.27 (t, J=7.1 Hz, 6H).

### Step (ii): Diethyl 3,4-dihydroxy-1-(4-methoxyphenyl)-1H-pyrrole-2,5-dicarboxylate (2)

Diethyl oxalate (83.0 mL, 0.610 mol) was added dropwise to a stirred solution of diethyl 2,2′-((4-methoxyphenyl)azanediyl)diacetate (1) (180 g, 0.610 mol) in NaOEt (21% by wt in EtOH) (506 mL, 1.30 mol), the mixture was stirred at 100° C. for 1 h. The reaction was quenched with acetic acid (210 mL, 3.70 mol) and the resulting suspension was poured into iced water (1 L), the resulting off-white solid collected by vacuum filtration. The crude product was recrystallised from hot EtOH (3.50 L) to give diethyl 3,4-dihydroxy-1-(4-methoxyphenyl)-1H-pyrrole-2,5-dicarboxylate (2) (152 g, 71%) as a white solid: m/z 350 (M+H)⁺ (ES⁺); 348 (M−H)⁻ (ES). ¹H NMR (400 MHz, DMSO-d₆) δ 8.64 (s, 2H), 7.13-7.01 (m, 2H), 6.92-6.81 (m, 2H), 3.99 (q, J=7.1 Hz, 4H), 3.78 (s, 3H), 0.99 (t, J=7.1 Hz, 6H).

### Step (iii): Diethyl 3,4-bis(benzyloxy)-1-(4-methoxyphenyl)-1H-pyrrole-2,5-dicarboxylate (3)

Benzyl bromide (42.6 mL, 358 mmol) was added dropwise to a stirred suspension of 3,4-dihydroxy-1-(4-methoxyphenyl)-1H-pyrrole-2,5-dicarboxylate (2) (50.0 g, 143 mmol) and K₂CO₃ (49.5 g, 358 mmol) in DMF (1 L), the reaction mixture was stirred at 60° C. for 4 h. After cooling to RT the reaction mixture was poured into ether (500 mL) and washed with brine (3×250 mL), dried (MgSO₄), filtered and concentrated in vacuo to afford a bright yellow solid. The crude product was triturated with isohexane to give diethyl 3,4-bis(benzyloxy)-1-(4-methoxyphenyl)-1H-pyrrole-2,5-dicarboxylate (3) (64.8 g, 85%) as a white solid: m/z 530 (M+H)⁺ (ES⁺). ¹H NMR (400 MHz, DMSO-d₆) δ: 7.48-7.29 (m, 10H), 7.17-7.09 (m, 2H), 6.95-6.87 (m, 2H), 5.09 (s, 4H), 3.99 (q, J=7.1 Hz, 4H), 3.80 (s, 3H), 0.99 (t, J=7.1 Hz, 6H).

### Step (iv): 3,4-Bis(benzyloxy)-1-(4-methoxyphenyl)-1H-pyrrole-2,5-dicarboxylic acid (4)

A mixture of diethyl 3,4-bis(benzyloxy)-1-(4-methoxyphenyl)-1H-pyrrole-2,5-dicarboxylate (3) (2.80 g, 5.29 mmol), 2M NaOH (aq.) (26.4 mL, 52.9 mmol), in ethanol (12 mL) and THF (20 mL) was stirred at 60° C. for 72 h. After cooling to RT, the mixture was acidified with 6M HCl_{(aq.)} and the resulting precipitate was collected by filtration, washed with water (5 mL), and Et₂O (5.00 mL) to afford 4-bis(benzyloxy)-1-(4-methoxyphenyl)-1H-pyrrole-2,5-dicarboxylic acid (4) (1.94 g, 67%) as an off-white solid: m/z 474 (M+H)⁺ (ES⁺); 472 (M−H)⁻ (ES). ¹H NMR (400 MHz, DMSO-d₆) δ 12.61 (s, 2H), 7.46-7.40 (m, 4H), 7.39-7.29 (m, 6H), 7.16-7.07 (m, 2H), 6.92-6.84 (m, 2H), 5.07 (s, 4H), 3.78 (s, 3H).

### Step (v): 3,4-Bis(benzyloxy)-N²,N²,N⁵,N⁵-tetramethyl-1-(4-methoxyphenyl)-1H-pyrrole-2,5-dicarboxamide (5)

To a solution of 3,4-bis(benzyloxy)-1-(4-methoxyphenyl)-1H-pyrrole-2,5-dicarboxylic acid (4)(110 mg, 0.232 mmol), and dimethylamine hydrochloride (56.8 mg, 0.697 mmol) in DMF (2 mL) at 0° C. was added DIPEA (243 μL, 1.39 mmol) and then immediately HATU (265 mg, 0.697 mmol) and the mixture stirred for 30 min. The reaction mixture was quenched with water and then partitioned between saturated aqueous ammonium chloride (20 mL) and ether (30 mL). The ether layer was taken and washed with further saturated ammonium chloride_{(aq.)} (15 mL) saturated aqueous sodium bicarbonate (2×15 mL), brine (15 mL) and then dried (MgSO₄), filtered and concentrated in vacuo to afford 3,4-bis(benzyloxy)-1-(4-methoxyphenyl)-N²,N²,N⁵,N⁵-tetramethyl-1H-pyrrole-2,5-dicarboxamide (5) (124 mg, 100%). m/z 528.3 (M+H)⁺ (ES⁺). ¹H NMR (400 MHz, CDCl₃) δ 7.37-7.25 (m, 10H), 7.11-7.06 (m, 2H), 6.82-6.77 (m, 2H), 5.06 (s, 4H), 3.76 (s, 3H), 2.79 (s, 6H), 2.63 (s, 6H).

### Step (vi): 3,4-Dihydroxy-1-(4-methoxyphenyl)-N²,N²,N⁵,N⁵-tetramethyl-1H-pyrrole-2,5-dicarboxamide (UL1-005)

3,4-Bis(benzyloxy)-1-(4-methoxyphenyl)-N²,N²,N⁵,N⁵-tetramethyl-1H-pyrrole-2,5-dicarboxamide (5) (5.00 g, 9.48 mmol) was dissolved in methanol (150 mL) and the solution was hydrogenated in the H-Cube (10% Pd/C, 70×4 mm, Full hydrogen, 40° C., 1 mL/min) then concentrated under vacuum. The resulting residue was recrystallised from isopropanol (100 mL) and dried in a desiccator to afford 3,4-dihydroxy-1-(4-methoxyphenyl)-N²,N²,N⁵,N⁵-tetramethyl-1H-pyrrole-2,5-dicarboxamide (UL1-005) (2.70 g, 78%) as a white crystalline solid: m/z 348.1 (M+H)⁺ (ES⁺); 346.0 (M−H)⁻ (ES). ¹H NMR (400 MHz, DMSO-d₆) δ: 8.38 (s, 2H), 6.94-6.89 (m, 2H), 6.86-6.81 (m, 2H), 3.73 (s, 3H), 2.88 (s, 12H).

### Example A2

### Alternative Potential Synthesis of 3,4-dihydroxy-1-(4-methoxyphenyl)-N²,N²,N⁵,N⁵-tetramethyl-1H-pyrrole-2,5-dicarboxamide (UL1-005)

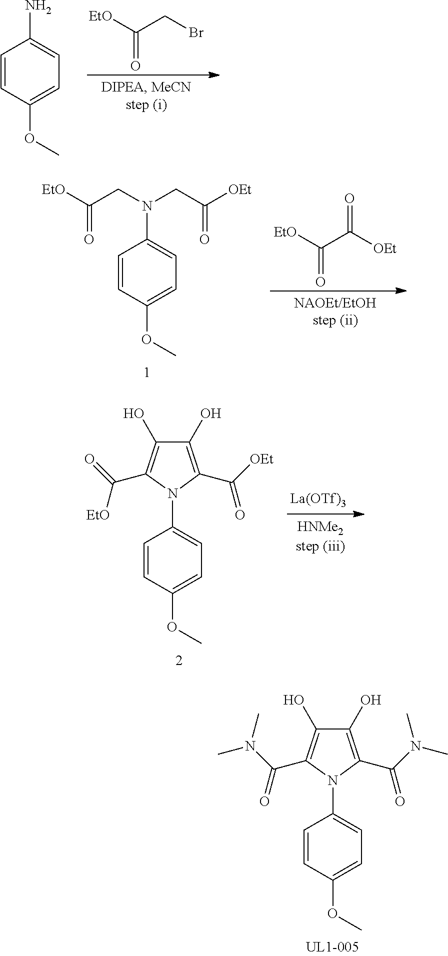

### Example B

### Ethyl 5-(dimethylcarbamoyl)-3,4-dihydroxy-1-(4-methoxyphenyl)-1H-pyrrole-2-carboxylate (UL1-012)

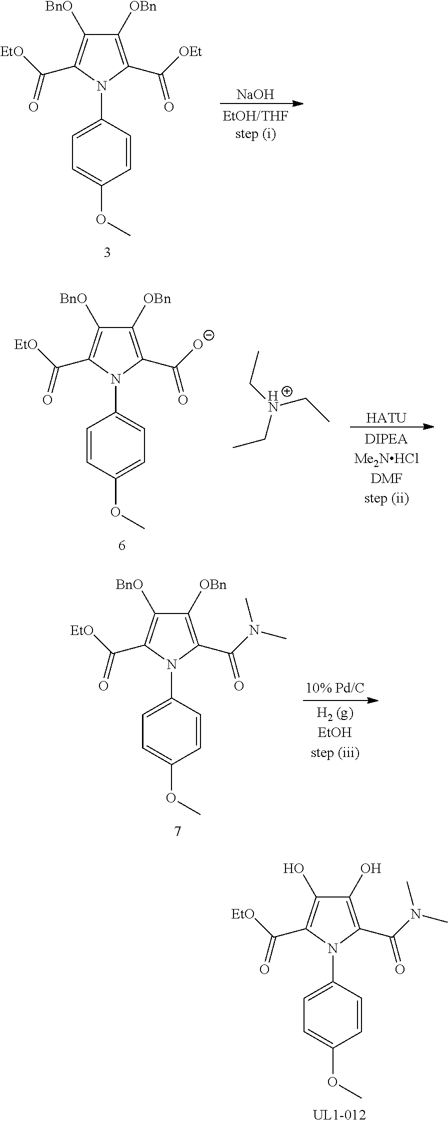

### Step (i): Triethylammonium 3,4-bis(benzyloxy)-5-(ethoxycarbonyl)-1-(4-methoxyphenyl)-1H-pyrrole-2-carboxylate (6)

To a solution of diethyl 3,4-bis(benzyloxy)-1-(4-methoxyphenyl)-1H-pyrrole-2,5-dicarboxylate (3) (39.6 g, 74.8 mmol) in THF/EtOH (300/50 mL) was added NaOH (3.07 g, 77 mmol) as a solution in water (20 mL). The reaction was stirred at 50° C. for 16 h. Triethylamine was added (30 mL, 215 mmol) and the volatiles were removed in vacuo. The residue was purified by silica gel chromatography (50% isohexane:DCM (+2% Et₃N), then 20% MeOH/EtOAc (+2% Et₃N)) to afford triethylammonium 3,4-bis(benzyloxy)-5-(ethoxycarbonyl)-1-(4-methoxyphenyl)-1H-pyrrole-2-carboxylate (6) (39.3 g, 83%) as a yellow oil: m/z 502 (M+H)⁺(ES⁺); 500 (M−H)⁻ (ES⁻). ¹H NMR (400 MHz, DMSO-d₆) δ: 7.51-7.26 (m, 10H), 7.11-7.05 (m, 2H), 6.92-6.83 (m, 2H), 5.09 (s, 2H), 5.06 (s, 2H), 3.95 (q, J=7.1 Hz, 2H), 3.79 (s, 3H), 2.85-2.62 (m, 6H), 1.08-0.92 (m, 12H).

### Step (ii): Ethyl 3,4-bis(benzyloxy)-5-(dimethylcarbamoyl)-1-(4-methoxyphenyl)-1H-pyrrole-2-carboxylate (7)

To a solution of triethylammonium 3,4-bis(benzyloxy)-5-(ethoxycarbonyl)-1-(4-methoxyphenyl)-1H-pyrrole-2-carboxylate (6) (10.84 g, 17.99 mmol) in DMF (150 mL), at 0° C. was added HATU (10.26 g, 27.0 mmol), dimethylamine hydrochloride (2.93 g, 36.0 mmol) and DIPEA (18.8 ml, 108 mmol). The reaction mixture was stirred at RT for 16 h and partitioned between EtOAc (500 mL) and 1M HCl (aq.) (250 mL). The organic phase was washed succesively with 1M HCl (aq.) (250 mL), sat. NaHCO₃ (aq.) (2×250 mL), and brine (2×250 mL), dried (MgSO₄), filtered and concentrated in vacuo to afford ethyl 3,4-bis(benzyloxy)-5-(dimethylcarbamoyl)-1-(4-methoxyphenyl)-1H-pyrrole-2-carboxylate (7) (7.62 g, 79%) as a light yellow oil, that solidified on standing: m/z 529 (M+H)⁺ (ES⁺). ¹H NMR (400 MHz, DMSO-d₆) δ: 7.51-7.21 (m, 10H), 7.14-7.03 (m, 2H), 6.94-6.84 (m, 2H), 5.12 (s, 2H), 4.96 (s, 2H), 4.00 (q, J=7.1 Hz, 2H), 3.77 (s, 3H), 2.70 (s, 6H), 1.00 (t, J=7.1 Hz, 6H).

### Step (iii): Ethyl 5-(dimethylcarbamoyl)-3,4-dihydroxy-1-(4-methoxyphenyl)-1H-pyrrole-2-carboxylate (UL1-012)

Ethyl 3,4-bis(benzyloxy)-5-(dimethylcarbamoyl)-1-(4-methoxyphenyl)-1H-pyrrole-2-carboxylate (7) (1.03 g, 1.94 mmol) was dissolved in EtOH and then treated with 10% Pd/C (37 mg). The reaction mixture was purged with N₂ for 5 min then Hydrogen gas was bubbled through the mixture with stirring at RT for 1.5 h. The mixture was filtered through Celite and concentrated in vacuo. The residual yellow solid was triturated with Et₂O to afford ethyl 5-(dimethylcarbamoyl)-3,4-dihydroxy-1-(4-methoxyphenyl)-1H-pyrrole-2-carboxylate (UL1-012) (602 mg, 89%) as a white solid: m/z 349 (M+H)⁺ (ES⁺), 347 (M−H)⁻ (ES⁻). ¹H NMR (400 MHz, DMSO-d₆) δ: 8.60 (s, 1H), 8.46 (s, 1H), 7.08-7.01 (m, 2H), 6.90-6.82 (m, 2H), 4.00 (q, J=7.0 Hz, 2H), 3.76 (s, 3H), 2.83 (br s, 6H), 0.99 (t, J=7.1 Hz, 6H).

### Example C

### 3,4-Dihydroxy-1-(4-methoxyphenyl)-N,N-dimethyl-5-(4-methylpiperazine-1-carbonyl)-1H-pyrrole-2-carboxamide (UL1-027)

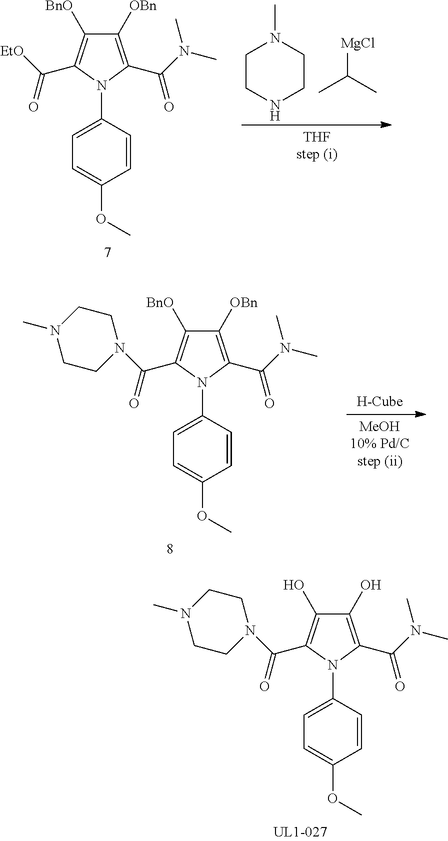

### Step (i): 3,4-bis(benzyloxy)-1-(4-methoxyphenyl)-N,N-dimethyl-5-(4-methylpiperazine-1-carbonyl)-1H-pyrrole-2-carboxamide (8)

To a stirred solution of ethyl 3,4-bis(benzyloxy)-5-(dimethylcarbamoyl)-1-(4-methoxyphenyl)-1H-pyrrole-2-carboxylate (7) (6.62 g, 12.5 mmol) and 1-methylpiperazine (3.18 ml, 25.1 mmol) in THF (100 mL) at 0° C. was added isopropylmagnesium chloride (15.7 ml, 31.3 mmol) dropwise. The reaction mixture was allowed to warm to RT and stirred for 1 h. The reaction was quenched with ammonium chloride_{(aq.)} (20 mL), diluted with brine (200 mL) and extracted with ethyl acetate (2×200 mL). The combined organic layers were dried (MgSO₄), filtered and concentrated in vacuo. The residue was triturated with diethyl ether (100 mL) and the solid was isolated by filtration, rinsing with diethyl ether, to afford 3,4-bis(benzyloxy)-1-(4-methoxyphenyl)-N,N-dimethyl-5-(4-methylpiperazine-1-carbonyl)-1H-pyrrole-2-carboxamide (8) (5.76 g, 79%) as a white solid: m/z 583 (M+H)⁺ (ES⁺). ¹H NMR (400 MHz, DMSO-d₆) δ: 7.40-7.30 (m, 10H), 7.03-6.98 (m, 2H), 6.95-6.90 (m, 2H), 5.00 (s, 4H), 3.76 (s, 3H), 3.41-3.30 (br m, 2H), 3.19-3.08 (br m, 2H), 2.74 (s, 3H), 2.72 (s, 3H), 2.14-2.00 (br m, 5H), 1.98-1.87 (br m, 2H).

### Step (ii): 3,4-dihydroxy-1-(4-methoxyphenyl)-N,N-dimethyl-5-(4-methylpiperazine-1-carbonyl)-1H-pyrrole-2-carboxamide (UL1-027)

A solution of 3,4-bis(benzyloxy)-1-(4-methoxyphenyl)-N,N-dimethyl-5-(4-methylpiperazine-1-carbonyl)-1H-pyrrole-2-carboxamide (8) (2.00 g, 3.43 mmol) in methanol (20 mL) was hydrogenated in the H-Cube (10% Pd/C, 55×4 mm, Full hydrogen, 40° C., 1 mL/min) and the reaction mixture was then concentrated in vacuo. The residue was triturated with diethyl ether (10 mL) and the solid was isolated by filtration, rinsing with diethyl ether, to afford 3,4-dihydroxy-1-(4-methoxyphenyl)-N,N-dimethyl-5-(4-methylpiperazine-1-carbonyl)-1H-pyrrole-2-carboxamide (UL1-027) (1.10 g, 2.68 mmol, 78% yield) as an off-white solid: m/z 403 (M+H)⁺ (ES⁺). ¹H NMR (400 MHz, DMSO-d₆) δ: 8.43 (br s, 2H), 6.95-6.90 (m, 2H), 6.88-6.83 (m, 2H), 3.74 (s, 3H), 3.44-3.36 (br m, 4H), 2.87 (s, 6H), 2.24-2.12 (br m, 7H).

### Example D

### 2,5-Bis(dimethylcarbamoyl)-1-(4-methoxyphenyl)-1H-pyrrole-3,4-diyl bis(2-methylpropanoate) (UL1-114)

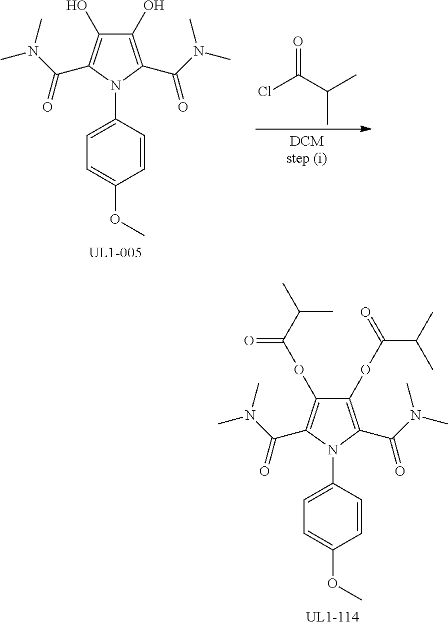

### Step (i): 2,5-Bis(dimethylcarbamoyl)-1-(4-methoxyphenyl)-1H-pyrrole-3,4-diylbis(2-methylpropanoate) (UL1-114)

To a stirred solution of 3,4-dihydroxy-1-(4-methoxyphenyl)-N²,N²,N⁵,N⁵-tetramethyl-1H-pyrrole-2,5-dicarboxamide (UL1-005) (0.065 g, 0.187 mmol) in acetonitrile (4 mL) at 0° C. was added isobutyryl chloride (0.043 mL, 0.412 mmol) followed by DIPEA (0.072 mL, 0.412 mmol). The reaction was allowed to reach RT and stirred for 3 h. The reaction mixture was then diluted with DCM (50 mL) and washed with water (50 mL) followed by brine (2×50 mL). The organic layer was dried (MgSO₄), filtered and concentrated in vacuo. The residue was purified by silica gel chromatography (40 g, 0-4% methanol in DCM) to afford a pale yellow oil. The product was further purified by preparative HPLC (Waters, Acidic (0.1% Formic acid), Waters X-Select Prep-C18, 5 μm, 19×50 mm column, 30-50% MeCN in Water) to afford 2,5-bis(dimethylcarbamoyl)-1-(4-methoxyphenyl)-1H-pyrrole-3,4-diyl bis(2-methylpropanoate) (UL1-114) (0.02 g, 22%) as a white solid: m/z 488 (M+H)⁺ (ES⁺). ¹H NMR (400 MHz, DMSO-d₆) δ: 7.12-7.08 (m, 2H), 6.97-6.93 (m, 2H), 3.77 (s, 3H), 2.85 (br s, 6H), 2.79-2.72 (m, 8H), 1.15 (d, J=7.0 Hz, 12 H).

### Example E

### 2-(Dimethylcarbamoyl)-1-(4-methoxyphenyl)-5-(4-methylpiperazine-1-carbonyl)-1H-pyrrole-3,4-diyl bis(2-methylpropanoate) hydrochloride (UL6-002)

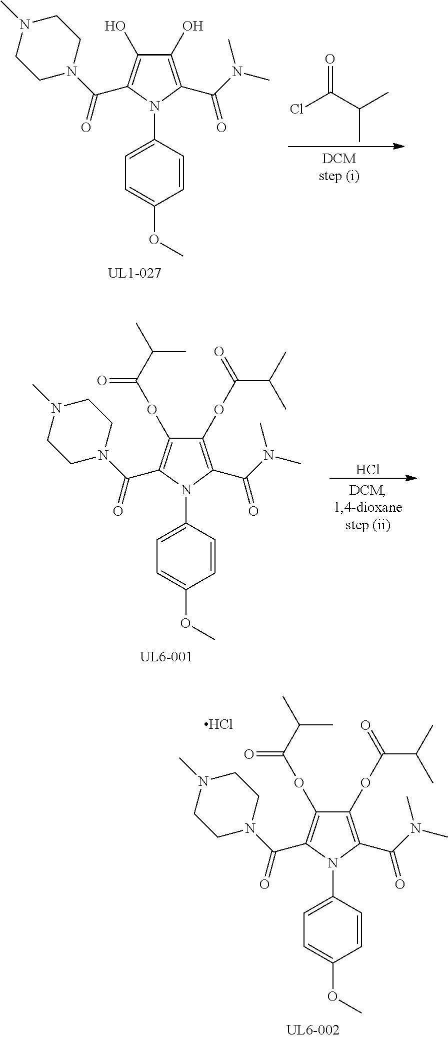

### Step (i): 2-(Dimethylcarbamoyl)-1-(4-methoxyphenyl)-5-(4-methylpiperazine-1-carbonyl)-1H-pyrrole-3,4-diyl bis(2-methylpropanoate) (UL6-001)

To a suspension of 2-tert-butylimino-2-diethylamino-1,3-dimethylperhydro-1,3,2-diazaphosphorine (polymer-bound,2.2 mmol/g) (1.10 g, 2.43 mmol) and 3,4-dihydroxy-1-(4-methoxyphenyl)-N,N-dimethyl-5-(4-methylpiperazine-1-carbonyl)-1H-pyrrole-2-carboxamide (UL1-027) (326 mg, 0.810 mmol) in DCM (5 mL) at 0° C. was added isobutyryl chloride (180 μL, 1.70 mmol) and the mixture allowed to warm to ambient temperature and shaken for 30 min. After this time it was filtered, solvents removed under reduced pressure and the resulting yellow oil purified by silica gel chromatography (40 g, 0-5% MeOH in DCM) to afford 2-(dimethylcarbamoyl)-1-(4-methoxyphenyl)-5-(4-methylpiperazine-1-carbonyl)-1H-pyrrole-3,4-diyl bis(2-methylpropanoate) (UL6-001) (176 mg, 38%) as a yellow oil. m/z 543 (M+H)⁺ (ES⁺). ¹H NMR (400 MHz, DMSO-d₆) δ: 7.13-7.08 (m, 2H), 7.00-6.95 (m, 2H), 3.78 (s, 3H), 3.40-3.17 (m, 4H), 2.87-2.71 (m, 8H), 2.21-1.96 (m, 7H), 1.17 (d, J=2.5 Hz, 6H), 1.15 (d, J=2.5 Hz, 6H).

### Step (ii): 2-(Dimethylcarbamoyl)-1-(4-methoxyphenyl)-5-(4-methylpiperazine-1-carbonyl)-1H-pyrrole-3,4-diyl bis(2-methylpropanoate) hydrochloride (UL6-002)

To a solution of 2-(dimethylcarbamoyl)-1-(4-methoxyphenyl)-5-(4-methylpiperazine-1-carbonyl)-1H-pyrrole-3,4-diylbis(2-methylpropanoate) (UL6-001) (88 mg, 0.154 mmol) in ether (5 mL) was added 4M HCl in dioxane (38.5 μL, 0.154 mmol) whereupon a yellow precipitate formed. Isohexanes (2 mL) were added to aid further precipitation and the resulting mixture spun down in Genevac (no vacuum) and supernatant removed by pipette. Added further ether (5 mL) and repeated spin and supernatant removal, removed remaining solvent under reduced pressure to afford 2-(dimethylcarbamoyl)-1-(4-methoxyphenyl)-5-(4-methylpiperazine-1-carbonyl)-1H-pyrrole-3,4-diylbis(2-methylpropanoate) hydrochloride (UL6-002) (92 mg, 98%) as a yellow powder: m/z 543 (M+H)⁺ (ES⁺). ¹H NMR (400 MHz, DMSO-d₆) δ: 10.61 (br s, 1H), 7.13-7.09 (m, 2H), 7.00-6.95 (m, 2H), 4.50-4.08 (br m, 2H) 3.78 (s, 3H), 3.50-3.19 (m, 3H), 2.87-2.54 (m, 14H), 1.17 (d, J=2.5 Hz, 6H), 1.15 (d, J=2.5 Hz, 6H).

### Example F

### 2-(Dimethylcarbamoyl)-1-(4-methoxyphenyl)-5-(4-methylpiperazine-1-carbonyl)-1H-pyrrole-3,4-diyl bis(2,2-dimethylpropanoate) hydrochloride (UL6-004)

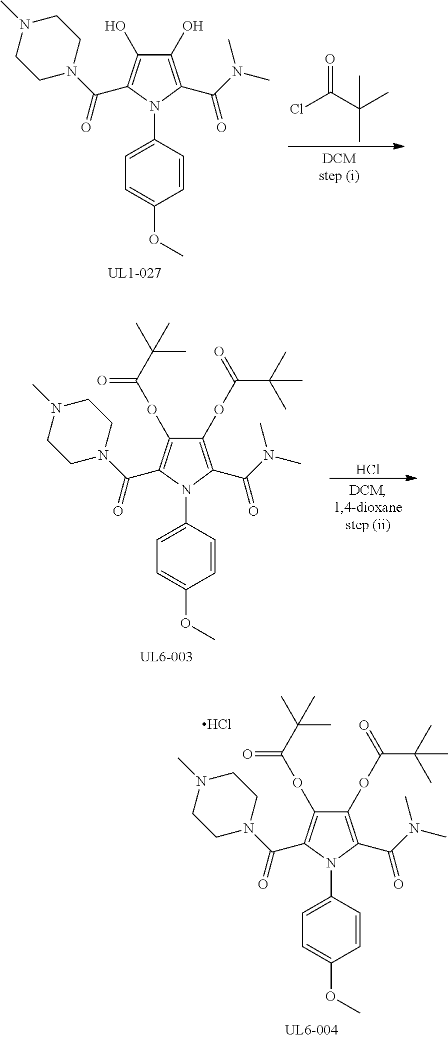

### Step (i): 2-(Dimethylcarbamoyl)-1-(4-methoxyphenyl)-5-(4-methylpiperazine-1-carbonyl)-1H-pyrrole-3 ,4-diyl bis(2,2-di methylpropanoate) (UL6-003)

To a solution/suspension of 3,4-dihydroxy-1-(4-methoxyphenyl)-N,N-dimethyl-5-(4-methylpiperazine-1-carbonyl)-1H-pyrrole-2-carboxamide (UL1-127) (1.07 g, 2.65 mmol) and 2-tert-butylimino-2-diethylamino-1,3-dimethylperhydro-1,3,2-diazaphosphorine (polymer-bound.2.2 mmol/g) (3.62 g, 7.96 mmol) in DCM (20 mL) at 0° C. was added pivaloyl chloride (0.692 mL, 5.84 mmol). The reaction mixture was allowed to warm to RT and shaken for 1 h.

The mixture was then filtered and concentrated in vacuo. The residue was triturated with diethyl ether (20 mL) and the resulting solid was isolated by fitration, rinsing with ether, to afford 2-(dimethylcarbamoyl)-1-(4-methoxyphenyl)-5-(4-methylpiperazine-1-carbonyl)-1H-pyrrole-3,4-diyl bis(2,2-dimethylpropanoate) (UL6-003) (1.01 g, 65%) as an off-white solid: m/z 571 (M+H)⁺ (ES⁺). ¹H NMR (400 MHz, DMSO-d₆) δ: 7.14-7.09 (m, 2H), 7.01-6.96 (m, 2H), 3.78 (s, 3H), 3.45-3.10 (br m, 4H), 2.86 (s, 3H), 2.76 (s, 3H), 2.32-1.87 (br m, 7H), 1.23 (s, 9H), 1.22 (s, 9H).

### Step (ii): 2-(Dimethylcarbamoyl)-1-(4-methoxyphenyl)-5-(4-methylpiperazine-1-carbonyl)-1H-pyrrole-3,4-diyl bis(2,2-dimethylpropanoate) hydrochloride (UL6-004)

To a solution of 2-(dimethylcarbamoyl)-1-(4-methoxyphenyl)-5-(4-methylpiperazine-1-carbonyl)-1H-pyrrole-3,4-diylbis(2,2-dimethylpropanoate) (UL6-003) (500 mg, 0.876 mmol) in DCM (10 mL) at 0° C. was added 4M HCl in dioxane (0.230 mL, 0.920 mmol). The reaction mixture was allowed to warm to RT then concentrated in vacuo. The residue was triturated with ethyl acetate (10 mL) and the solid was isolated by filtration, rinsing with ethyl acetate, to afford 2-(dimethylcarbamoyl)-1-(4-methoxyphenyl)-5-(4-methylpiperazine-1-carbonyl)-1H-pyrrole-3,4-diyl bis(2,2-dimethylpropanoate) hydrochloride (UL6-004) (0.436 g, 82%) as an off white solid: m/z 571 (M+H)⁺ (ES⁺). ¹H NMR (400 MHz, DMSO-d₆) δ: 10.94 (br s, 1H), 7.20-7.14 (m, 2H), 7.02-6.96 (m, 2H), 4.45-3.95 (br m, 2H), 3.79 (s, 3H), 3.52-3.28 (br m, 3H), 3.15-2.53 (m, 12H), 1.25 (s, 9H), 1.23 (s, 9H).

### Example G

### Sodium ((((2,5-bis(dimethylcarbamoyl)-1-(4-methoxyphenyl)-1H-pyrrole-3,4-diyl)bis(oxy))bis(carbonyl))bis(3,1-phenylene))bis(methylene) bis(hydrogenphosphate) (UL6-006)

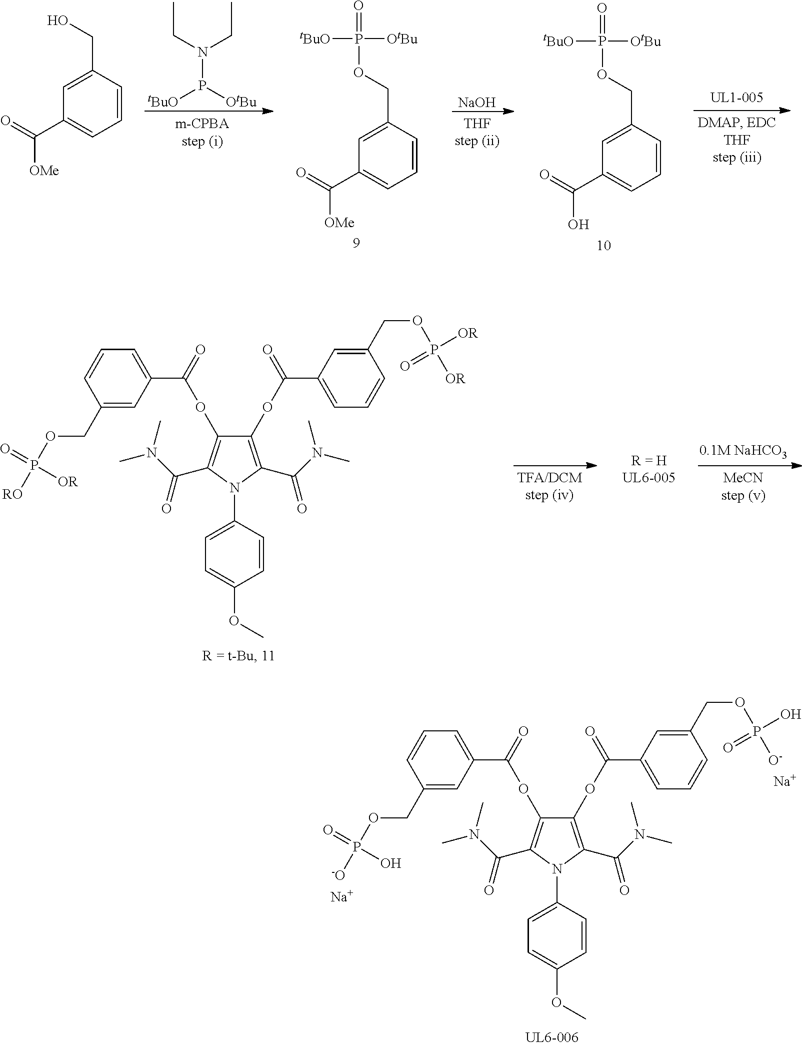

### Step (i): Methyl 3-(((di-tert-butoxyphosphoryl)oxy)methyl)benzoate (9)

To a solution of methyl 3-(hydroxymethyl)benzoate (3.00 g, 18.1 mmol) and di-tert-butyl diethylphosphoramidite (6.75 g, 27.1 mmol) in THF (100 mL) was added 5-methyl-1H-tetrazole (3.04 g, 36.1 mmol). The reaction mixture was stirred at RT for 4 h 30 mins, then cooled to −78° C. for 10 mins before the addition of 3-chlorobenzoperoxoic acid (7.28 g, 32.5 mmol). The reaction mixture warmed to rt and stirred for 1 h at RT, then quenched by the addition of sodium 10 bisulphite_{(aq.)} (˜40%, 50 mL). The volatiles were removed in vacuo and the aqueous residue was partitioned between ethyl acetate (200 mL) and water (100 mL). The aqueous phase was extracted with a further portion of ethyl acetate (100 mL). Combined organics were washed sequentially with sodium hydrogen carbonate_{(aq.)} (3×150 mL) and brine (100 mL), dried (MgSO₄) and concentrated in vacuo to afford a pale yellow oil. The oil was dissolved in ethyl acetate (200 mL) and washed sequentially with sodium hydrogen carbonate_{(aq.)} (4×150 mL) and brine (100 mL), dried (MgSO₄) and concentrated in vacuo to afford a pale yellow oil. The crude material was purified by silica gel chromatography (120 g, 0-100% ethyl acetate in isohexanes) to afford methyl 3-(((di-tert-butoxyphosphoryl)oxy)methyl)benzoate (9) (5.58 g, 81%): m/z 381 (M+Na)⁺ (ES⁺).¹H NMR (400 MHz, DMSO-d₆) δ: 8.01 (td, J=1.7, 0.7 Hz, 1H), 7.92 (dt, J=7.8, 1.5 Hz, 1H), 7.66 (ddd, J=7.7, 1.9, 1.1 Hz, 1H), 7.55 (dd, J=8.0, 7.4 Hz, 1H), 5.01 (d, J=8.3 Hz, 2H), 3.86 (s, 3H), 1.44-1.36 (m, 18H).

### Step (ii): 3-(((Di-tert-butoxyphosphoryl)oxy)methyl)benzoic acid (10)

Methyl 3-(((di-tert-butoxyphosphoryl)oxy)methyl)benzoate (9) (3.00 g, 8.37 mmol) was dissolved in THF (30 mL). Sodium hydroxide (0.670 g, 16.7 mmol) was dissolved in water (3 mL) and the solution added to the reaction mixture followed by ethanol (3 mL).The reaction mixture was left to stir at RT for 18 h, then the solvents removed in vacuo. Water (20 mL) was added to the residue to afford a solution, which was acidified by the dropwise addition of 1M phosphoric acid. The precipitated solution was extracted with ethyl acetate (2×50 mL). Combined organics were washed with brine (30 mL), dried (MgSO₄) and concentrated in vacuo to afford 3-(((di-tert-butoxyphosphoryl)oxy)methyl)benzoic acid (10) (2.38 g, 75%) as an off white solid: m/z 343 (M−H)⁻ (ES⁻). ¹H NMR (400 MHz, DMSO-d₆) δ: 13.01 (s, 1H), 8.00 -7.97 (m, 1H), 7.90 (dt, J=7.8, 1.5 Hz, 1H), 7.65-7.59 (m, 1H), 7.52 (t, J=7.7 Hz, 1H), 5.00 (d, J=8.2 Hz, 2H), 1.45-1.33 (m, 18H).

### Step (iii): 2,5-Bis(dimethylcarbamoyl)-1-(4-methoxyphenyl)-1H-pyrrole-3,4-diyl bis(3-(((di-tert-butoxyphosphoryl)oxy) methyl)benzoate) (11)

To a stirred solution of 3,4-dihydroxy-1-(4-methoxyphenyl)-N²,N²,N⁵,N⁵-tetramethyl-1H-pyrrole-2,5-dicarboxamide (UL1-005) (857 mg, 2.47 mmol), N,N-dimethylpyridin-4-amine (121 mg, 0.987 mmol) and 3-(((di-tert-butoxyphosphoryl)oxy)methyl)benzoic acid (10) (2.38 g, 6.91 mmol) in THF (40 mL) was added N¹-((ethylimino)methylene)-N³,N³-dimethylpropane-1,3-diamine (1.22 mL, 6.91 mmol) and the reaction was stirred at RT for 2 h. The mixture was poured into saturated ammonium carbonate solution (50 mL) and extracted with ethyl acetate (50 mL) followed by DCM (2×50 mL). The combined organic layers were dried over (MgSO₄), filtered and concentrated in vacuo. The crude product was purified by silica gel chromatography (80 g, 10% THF in DCM) to afford 2,5-bis(dimethylcarbamoyl)-1-(4-methoxyphenyl)-1H-pyrrole-3,4-diyl bis(3-(((di-tert-butoxyphosphoryl)oxy)methyl)benzoate) (11) (0.645 g, 26%) as a pale yellow solid: ¹H NMR (400 MHz, DMSO-d₆) δ: 8.03 (s, 2H), 7.95 (d, J=7.9 Hz, 2H), 7.71 (d, J=7.9 Hz, 2H), 7.57 (t, J=7.9 Hz, 2H), 7.22-7.17 (m, 2H), 7.02-6.97 (m, 2H), 4.99 (d, J=8.6 Hz, 4H), 3.80 (s, 3H), 2.88 (s, 6H), 2.73 (s, 6H), 1.35 (s 36H).

### Step (iv): 2,5-bis(dimethylcarbamoyl)-1-(4-methoxyphenyl)-1H-pyrrole-3,4-diyl bis(3-40 ((phosphonooxy)methyl)benzoate) (UL6-005)

To a stirred solution of 2,5-bis(dimethylcarbamoyl)-1-(4-methoxyphenyl)-1H-pyrrole-3,4-diyl bis(3-(((di-tert-butoxyphosphoryl)oxy)methyl)benzoate) (11) (642 mg, 0.642 mmol) in DCM (25 mL) was added TFA (2.5 mL, 32.4 mmol). After 30 minutes, the reaction mixture was concentrated in vacuo and triturated with diethyl ether (20 mL). The solid was isolated by filtration and dried in vacuo to afford 2,5-bis(dimethylcarbamoyl)-1-(4-methoxyphenyl)-1H-pyrolle-3,4-diyl bis(3-((phosphonooxy)methyl)benzoate) (UL6-005) (288 mg, 57%) as a white solid: ¹H NMR (400 MHz, DMSO-d6) δ: 8.00 (s, 2H), 7.93 (d, J=7.9 Hz, 2H), 7.71 (d, J=7.9 Hz, 2H), 7.56 (t, J=7.9 Hz, 2H), 7.22-7.18 (m, 2H), 7.02-6.98 (m, 2H), 4.95 (d, J=7.5 Hz, 4H), 3.80 (s, 3H), 2.89 (s, 6H), 2.75 (s, 6H).

### Step (v): Sodium ((((2,5-bis(dimethylcarbamoyl)-1-(4-methoxyphenyl)-1H-pyrrole-3,4-diyl)bis(oxy))bis(carbonyl))bis(3,1-phenylene))bis(methylene) bis(hydrogenphosphate) (UL6-006)

To a suspension of 2,5-bis(dimethylcarbamoyl)-1-(4-methoxyphenyl)-1H-pyrrole-3,4-diyl bis(3-((phosphonooxy)methyl)benzoate) (UL6-005) (277 mg, 0.357 mmol) in acetonitrile (2 mL) was added 0.1M sodium bicarbonate (7.14 mL, 0.714 mmol). The resulting solution was allowed to stand for 1 h then the acetonitrile was removed in vacuo. The resulting aqueous solution was then freeze-dried to afford sodium ((((2,5-bis(dimethylcarbamoyl)-1-(4-methoxyphenyl)-1H-pyrrole-3,4-diyl)bis(oxy))bis(carbonyl))bis(3,1-phenylene))bis(methylene) bis(hydrogenphosphate) (UL6-006) (280 mg, 95%) as a white powder: m/z 776.0 (M+3H)⁺ (ES⁺). 1H NMR (400 MHz, D2O) δ: 8.13 (s, 2H), 8.07 (d, J=7.9 Hz, 2H), 7.80 (d, J=7.9 Hz, 2H), 7.58 (t,2H) 7.34-7.30 (m, 2H), 7.14-7.10 (m, 2H), 4.96 (d, J=7.1 Hz, 4H), 3.91 (s, 3H), 3.11 (s, 6H), 2.90 (s, 6H).

### Example H

### Sodium ((((2-(dimethylcarbamoyl)-5-(ethoxycarbonyl)-1-(4-methoxyphenyl)-1H-pyrrole-3,4-diAbis(oxy))bis(carbonyl))bis(4,1-phenylene))bis(methylene) bis(hydrogenphosphate) (UL6-008)

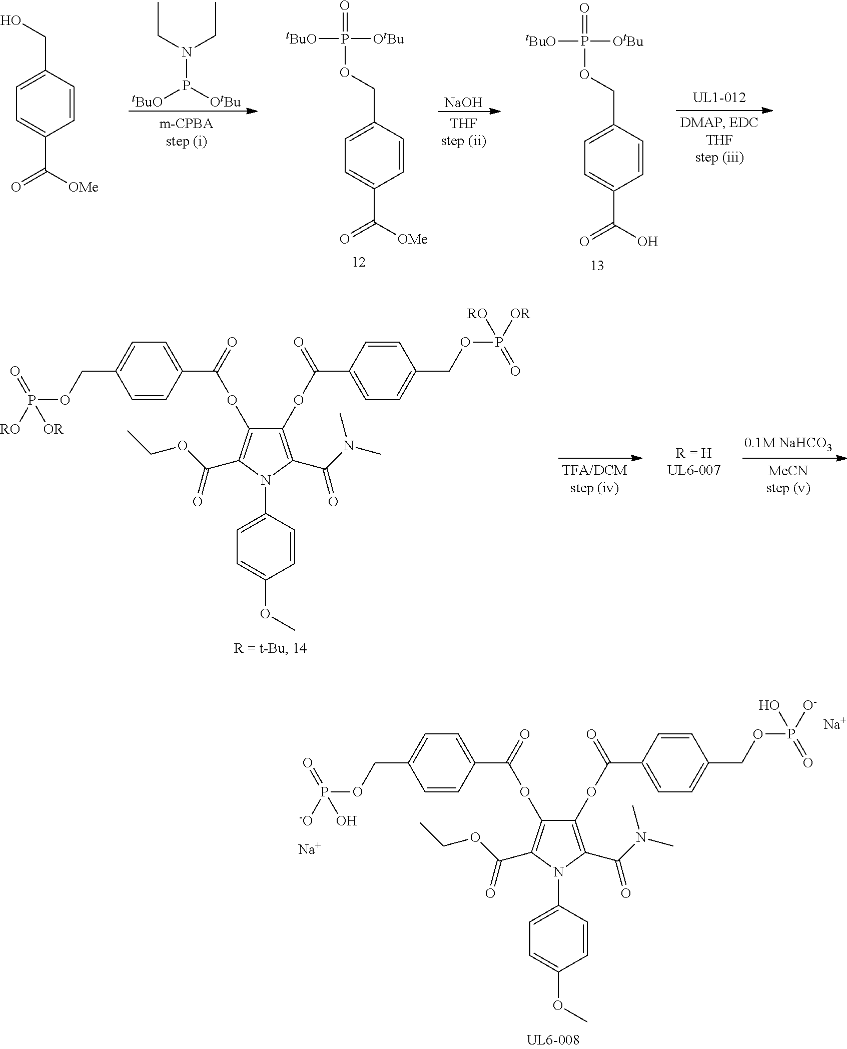

### Step (i): Methyl 4-(((di-tert-butoxyphosphoryl)oxy)methyl)benzoate (12)

To a stirred solution of methyl 4-(hydroxymethyl)benzoate (5.00 g, 30.1 mmol) and di-tert-butyl diethylphosphoramidite (12.56 mL, 45.1 mmol) in THF (150 mL) was added 5-methyl-1H-tetrazole (2.53 g, 30.1 mmol) and the reaction was stirred at RT. After 4 h, the reaction mixture was cooled to −78° C., and 3-chlorobenzoperoxoic acid (12.1 g, 54.2 mmol) was added. The mixture was allowed to warm to RT and stirred for 16 h. The reaction mixture was quenched with saturated sodium bisulphite solution (100 mL) and extracted with ethyl acetate (2×200 mL). The combined organic layers were washed with saturated sodium bicarbonate_{(aq.)} (500 mL), dried (MgSO₄), filtered and concentrated in vacuo. The crude product was purified by silica gel chromatography (330 g, 0-100% ethyl acetate in hexane) to afford methyl 4-(((di-tert-butoxyphosphoryl)oxy)methyl)benzoate (12) (9.32 g, 86%) as a white solid: m/z 381.0 (M+Na)⁺ (ES⁺). ¹H NMR (400 MHz, DMSO-d₆) δ: 7.99-7.97 (m, 2H), 7.54-7.51 (m, 2H), 5.01 (d, J=8.3 Hz, 2H), 3.85 (s, 3H), 1.44-1.37 (m, 18H).

### Step (ii): 4-(((Di-tert-butoxyphosphoryl)oxy)methyl)benzoic acid (13)

To a stirred solution of methyl 4-(((di-tert-butoxyphosphoryl)oxy)methyl)benzoate (12) (1.63 g, 4.55 mmol) in THF (20 mL) was added sodium hydroxide (364 mg, 9.10 mmol) in water (4 mL) followed by ethanol (4 mL). The reaction mixture was then stirred at RT for 16 h. The organic solvents were then removed in vacuo and the resulting aqueous solution was acidifid to ca. pH 6 by dropwise addition of 1M phosphoric acid. The solution was was then extracted with DCM (2×10.0 ml) and the combined organic layers were dried (MgSO₄), filtered and concentrated in vacuo. The resulting residue was triturated with ethyl acetate (10 mL) and isolated by filtration to afford 4-(((di-tert-butoxyphosphoryl)oxy)methyl)benzoic acid (13) (1.01 g, 61%) as a white solid: m/z 367.0 (M+Na)⁺ (ES⁺); 343.0 (M−H)⁻ (ES). ¹H NMR (400 MHz, DMSO-d₆) δ: 12.99 (s, 1H), 7.98-7.94 (m, 2H), 7.51-7.47 (m, 2H), 5.00 (d, J=8.2 Hz, 2H), 1.41 (s, 18H).

### Step (iii): 2-(Dimethylcarbamoyl)-5-(ethoxycarbonyl)-1-(4-methoxyphenyl)-1H-pyrrole-3,4-diyl bis(4-(((di-tert-butoxyphosphoryl)oxy)methyl)benzoate) (14)

To a stirred solution of 4-(((di-tert-butoxyphosphoryl)oxy)methyl)benzoic acid (13) (2.53 g, 7.35 mmol), ethyl 5-(dimethylcarbamoyl)-3,4-dihydroxy-1-(4-methoxyphenyl)-1H-pyrrole-2-carboxylate (UL1-012) (914 mg, 2.63 mmol) and N,N-dimethylpyridin-4-amine (128 mg, 1.05 mmol) in THF (60 mL) was added N¹-((ethylimino)methylene)-N³,N³-dimethylpropane-1,3-diamine (1.30 mL, 7.35 mmol) and the reaction was stirred at RT for 24 h. The reaction mixture was poured into saturated ammonium chloride_{(aq.)} (100 mL) and extracted with ethyl acetate (100 mL) followed by DCM (2×100 mL). The combined organic layers were dried (MgSO₄), filtered and concentrated in vacuo. The crude product was purified by silica gel chromatography (120 g, ethyl acetate) to afford 2-(dimethylcarbamoyl)-5-(ethoxycarbonyl)-1-(4-methoxyphenyl)-1H-pyrrole-3,4-diyl bis(4-(((di-tert-butoxyphosphoryl)oxy)methyl)-benzoate) (14) (1.19 g, 43%) as a white solid: ¹H NMR (400 MHz, DMSO-d₆) δ: 8.07-8.00 (m, 4H), 7.55 (dd, J=8.5, 3.5 Hz, 4H), 7.32-7.27 (m, 2H), 7.01-6.96 (m, 2H), 5.02 (d, J=8.2 Hz, 2H), 5.01 (d, J=8.0 Hz, 2H), 3.93 (q, J=7.2 Hz, 2H), 3.81 (s, 3H), 2.89 (s, 3H), 2.69 (s, 3H), 1.38 (s, 36H), 0.81 (t, J=7.2 Hz, 3H).

### Step (iv): 2-(Dimethylcarbamoyl)-5-(ethoxycarbonyl)-1-(4-methoxyphenyl)-1H-pyrrole-3,4-diyl bis(4-((phosphonooxy)methyl)benzoate) (U L6-007)

To a stirred solution of 2-(dimethylcarbamoyl)-5-(ethoxycarbonyl)-1-(4-methoxyphenyl)-1H-pyrrole-3,4-diyl bis(4-(((di-tert-butoxyphosphoryl)oxy)methyl)benzoate) (14) (1.15 g, 1.149 mmol) in DCM (50 ml) was added trifluoroacetic acid (2.5 ml, 32.4 mmol) and the reaction mixture was stirred at RT for 1 h. The reaction mixture was concentrated in vacuo. The residue was triturated with acetic acid (20 mL) and the resultant solid was filtered, rinsing with acetic acid and diethyl ether and freeze-dried to afford 2-(dimethylcarbamoyl)-5-(ethoxycarbonyl)-1-(4-methoxyphenyl)-1H-pyrrole-3,4-diyl bis(4-((phosphonooxy)methyl)benzoate) (U L6-007) (0.516 g, 57%) as a white solid: m/z 777 (M+H)⁺ (ES⁺). ¹H NMR (400 MHz, DMSO-d₆) δ: 8.06-7.99 (m, 4H), 7.57-7.51 (m, 4H), 7.32-7.27 (m, 2H), 7.01-6.96 (m, 2H), 4.98 (d, J=7.5 Hz, 2H), 4.97 (d, J=7.6 Hz, 2H), 3.94 (q, J=7.2 Hz, 2H), 3.81 (s, 3H), 2.89 (s, 3H), 2.70 (s, 3H), 0.82 (t, J=7.2 Hz, 3H).

### Step (v): Sodium ((((2-(dimethylcarbamoyl)-5-(ethoxycarbonyl)-1-(4-methoxyphenyl)-1H-pyrrole-3,4-diyl)bis(oxy))bis(carbonyl))bis(4,1-phenylene))bis(methylene) bis(hydrogenphosphate) (UL6-008)

To a solution of 2-(dimethylcarbamoyl)-5-(ethoxycarbonyl)-1-(4-methoxyphenyl)-1H-pyrrole-3,4-diyl bis(4-((phosphonooxy)methyl)benzoate) (UL6-007) (0.516 g, 0.664 mmol) in water (10 ml) and acetonitrile (10 ml) was added 0.1M sodium bicarbonate_{(aq.)} (13.3 ml, 1.33 mmol). The solution was allowed to stand for 20 minutes then the acetonitrile was removed under vacuum. The resulting aqueous solution was then freeze dried to afford sodium ((((2-(dimethylcarbamoyl)-5-(ethoxycarbonyl)-1-(4-methoxyphenyl)-1H-pyrrole-3,4-diyl)bis(oxy))bis(carbonyl))bis(4,1-phenylene))bis(methylene) bis(hydrogenphosphate) (UL6-008) (0.537 g, 98%) as a white solid: m/z 777 (M+3H)⁺ (ES⁺). ¹H NMR (400 MHz, DMSO-D₂O) δ: 8.14-8.09 (m, 4H), 7.57 (d, J=8.2 Hz, 4H), 7.44-7.39 (m, 2H), 7.15 (m, 2H), 5.02-4.96 (m, 4H), 4.02 (q, J=7.2 Hz, 2H), 3.93 (s, 3H), 3.10 (s, 3H), 2.83 (s, 3H), 0.82 (t, J=7.2 Hz, 3H).

The following Examples in Table 1 were prepared using the methods above.

Entries 1-4 are provided for comparative purposes and are not claimed as an aspect of the invention.

TABLE 1

| | Structure | | IC₅₀ | ¹H NMR Data | |
|---|---|---|---|---|---|
| Entry | (preparation method) | Compound name | (μM) | (DMSO-d₆) | Ionisation |
| 1* | 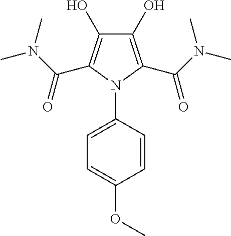 UL1-005 (Example A) | 3,4-Dihydroxy-1-(4- methoxyphenyl)- N²,N²,N⁵,N⁵-tetramethyl- 1H-pyrrole-2,5- dicarboxamide | 0.2 | δ: 8.38 (s, 2H), 6.94-6.89 (m, 2H), 6.86-6.81 (m, 2H), 3.73 (s, 3H), 2.88 (s, 12H) | m/z 348.1 (M + H)⁺ (ES⁺); 346.0 (M − H)⁻ (ES⁻) |
| 2* | 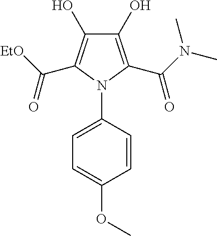 [SMILES: CCOC(=O)c1c(O)c(O)c(C(=O)N(C)C)n1-c1ccc(OC)cc1] UL1-012 (Example B) | Ethyl 5- (dimethylcarbamoyl)-3,4- dihydroxy-1-(4- methoxyphenyl)-1H- pyrrole-2-carboxylate | 0.2 | δ: 8.60 (s, 1H), 8.46 (s, 1H), 7.08- 7.01 (m, 2H), 6.90- 6.82 (m, 2H), 4.00 (q, J = 7.0 Hz, 2H), 3.76 (s, 3H), 2.83 (br s, 6H), 0.99 (t, J = 7.1 Hz, 6H) | m/z 349 (M + H)⁺ (ES⁺), 347 (M − H)⁻ (ES⁻) |
| 3* | 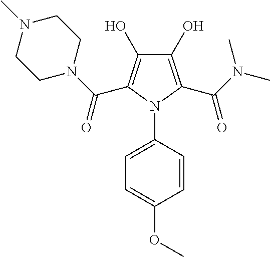 UL1-027 (Example C) | 3,4-Dihydroxy-1-(4- methoxyphenyl)-N,N- dimethyl-5-(4- methylpiperazine-1- carbonyl)-1H-pyrrole-2- carboxamide | 0.2 | δ: 8.43 (br s, 2H), 6.95-6.90 (m, 2H), 6.88-6.83 (m, 2H), 3.74 (s, 3H), 3.44- 3.36 (br m, 4H), 2.87 (s, 6H), 2.24- 2.12 (br m, 7H) | m/z 403 (M + H)⁺ (ES⁺) |
| 4 | 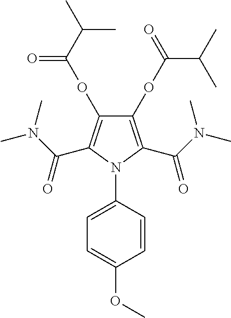 UL1-114 (Example D) | 2,5-Bis(dimethylcarbamoyl)- 1-(4-methoxyphenyl)-1H- pyrrole-3,4-diyl bis(2- methylpropanoate) | 19.5 | δ: 7.12-7.08 (m, 2H), 6.97-6.93 (m, 2H), 3.77 (s, 3H), 2.85 (br s, 6H), 2.79-2.72 (m, 8H), 1.15 (d, J = 7.0 Hz, 12 H). | m/z 488 (M + H)⁺ (ES⁺) |
| 5 | 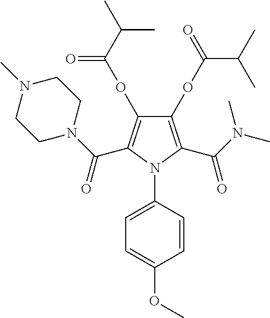 UL6-001 (Example E) | 2-(Dimethylcarbamoyl)-1- (4-methoxyphenyl)-5-(4- methylpiperazine-1- carbonyl)-1H-pyrrole-3,4- diyl bis(2- methylpropanoate) | Not tested | δ: 7.13-7.08 (m, 2H), 7.00-6.95 (m, 2H), 3.78 (s, 3H), 3.40-3.17 (m, 4H), 2.87-2.71 (m, 8H), 2.21-1.96 (m, 7H), 1.17 (d, J = 2.5 Hz, 6H), 1.15 (d, J = 2.5 Hz, 6H). | m/z 543 (M + H)⁺ (ES⁺) |
| 6 | 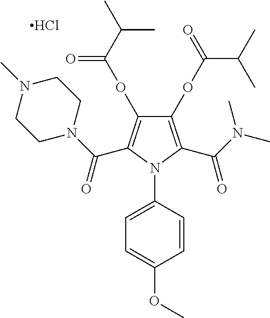 UL6-002 (Example E) | 2-(Dimethylcarbamoyl)-1- (4-methoxyphenyl)-5-(4- methylpiperazine-1- carbonyl)-1H-pyrrole-3,4- diyl bis(2- methylpropanoate) hydrochloride | 2 | δ: 10.61 (br s, 1H), 7.13-7.09 (m, 2H), 7.00-6.95 (m, 2H), 4.50-4.08 (br m, 2H) 3.78 (s, 3H), 3.50-3.19 (m, 3H), 2.87-2.54 (m, 14H), 1.17 (d, J = 2.5 Hz, 6H), 1.15 (d, J = 2.5 Hz, 6H). | m/z 543 (M + H)⁺ (ES⁺) |
| 7 | 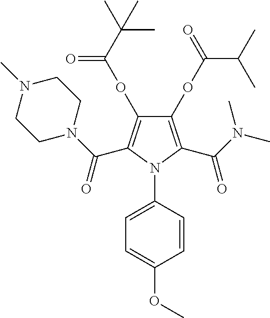 UL6-003 (Example F) | 2-(Dimethylcarbamoyl)-1- (4-methoxyphenyl)-5-(4- methylpiperazine-1- carbonyl)-1H-pyrrole-3,4- diyl bis(2,2- dimethylpropanoate) | Not tested | δ: 7.14-7.09 (m, 2H), 7.01-6.96 (m, 2H), 3.78 (s, 3H), 3.45-3.10 (br m, 4H), 2.86 (s, 3H), 2.76 (s, 3H), 2.32- 1.87 (br m, 7H), 1.23 (s, 9H), 1.22 (s, 9H) | m/z 571 (M + H)⁺ (ES⁺) |
| 8 | 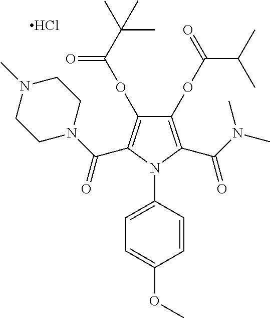 UL6-004 (Example F) | 2-(Dimethylcarbamoyl)-1- (4-methoxyphenyl)-5-(4- methylpiperazine-1- carbonyl)-1H-pyrrole-3,4- diyl bis(2,2- dimethylpropanoate) hydrochloride | 1.6 | δ: 10.94 (s, 1H), 7.20-7.14 (m, 2H), 7.02-6.96 (m, 2H), 4.45-3.95 (m, 2H), 3.79 (s, 3H), 3.52- 3.28 (m, 3H), 3.15- 2.53 (m, 12H), 1.25 (s, 9H), 1.23 (s, 9H). | m/z 571 (M + H)⁺ (ES⁺) |
| 9 | 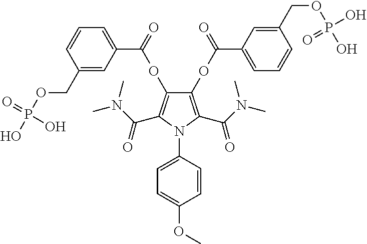 [SMILES: COc1ccc(-n2c(C(=O)N(C)C)c(OC(=O)c3cccc(COP(=O)(O)O)c3)c(OC(=O)c3cccc(COP(=O)(O)O)c3)c2C(=O)N(C)C)cc1] UL6-005 (Example G) | 2,5-Bis(dimethylcarbamoyl)- 1-(4-methoxyphenyl)-1H- pyrrole-3.4-diyl bis(3- ((phosphonooxy)methyl) benzoate) | Not tested | δ: 8.00 (s, 2H), 7.93 (d, J = 7.9 Hz, 2H), 7.71 (d, J = 7.9 Hz, 2H), 7.56 (t, J = 7.9 Hz, 2H), 7.22-7.18 (m, 2H), 7.02-6.98 (m, 2H), 4.95 (d, J = 7.5 Hz, 4H), 3.80 (s, 3H), 2.89 (s, 6H), 2.75 (s, 6H) | Retention time 1.09 min using ammonium acetate method of analytical HPLC |
| 10 | 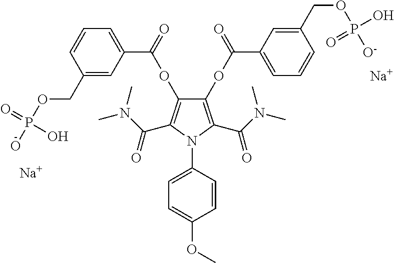 [SMILES: COc1ccc(-n2c(C(=O)N(C)C)c(OC(=O)c3cccc(COP(=O)([O-])O)c3)c(OC(=O)c3cccc(COP(=O)(O)O)c3)c2C(=O)N(C)C)cc1 ; [Na+] ; [Na+]] UL6-006 (Example G) | Sodium ((((2,5- bis(dimethylcarbamoyl)-1- (4-methoxyphenyl)-1H- pyrrole-3,4- diyl)bis(oxy))bis (carbonyl))bis(3,1- phenylene))bis(methylene) bis(hydrogenphosphate) | 0.3 | δ: 8.13 (s, 2H), 8.07 (d, J = 7.9 Hz, 2H), 7.80 (d, J = 7.9 Hz, 2H), 7.58 (t, 2H) 7.34- 7.30 (m, 2H), 7.14- 7.10 (m, 2H), 4.96 (d, J = 7.1 Hz, 4H), 3.91 (s, 3H), 3.11 (s, 6H), 2.90 (s, 6H) | m/z 776.0 (M + 3H)⁺ (ES⁺) |
| 11 | 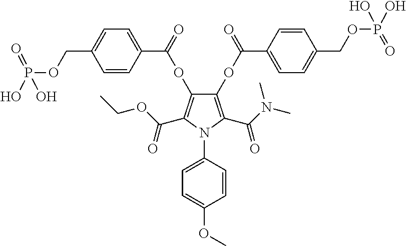 [SMILES: CCOC(=O)c1c(OC(=O)c2ccc(COP(=O)(O)O)cc2)c(OC(=O)c2ccc(COP(=O)(O)O)cc2)c(C(=O)N(C)C)n1-c1ccc(OC)cc1] UL6-007 (Example H) | 2-(Dimethylcarbamoyl)-5- (ethoxycarbonyl)-1-(4- methoxyphenyl)-1H-pyrrole- 3,4-diyl bis(4- ((phosphonooxy)methyl) benzoate) | Not tested | δ: 8.06-7.99 (m, 4H), 7.57-7.51 (m, 4H), 7.32-7.27 (m, 2H), 7.01-6.96 (m, 2H), 4.98 (d, J = 7.5 Hz, 2H), 4.97 (d, J = 7.6 Hz, 2H), 3.94 (q, J = 7.2 Hz, 2H), 3.81 (s, 3H), 2.89 (s, 3H), 2.70 (s, 3H), 0.82 (t, J = 7.2 Hz, 3H). | m/z 777 (M + H)⁺ (ES⁺) |
| 12 | 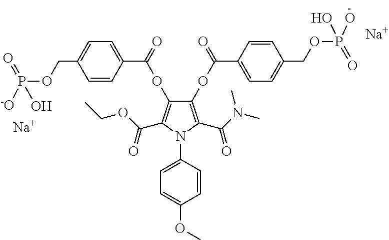 UL6-008 (Example H) | Sodium ((((2- (dimethylcarbamoyl)-5- (ethoxycarbonyl)-1-(4- methoxyphenyl)-1H-pyrrole- 3,4- diyl)bis(oxy))bis (carbonyl))bis(4,1- phenylene))bis(methylene) bis(hydrogenphosphate) | 1.9 | δ: 8.14-8.09 (m, 4H), 7.57 (d, J = 8.2 Hz, 4H), 7.44- 7.39 (m, 2H), 7.15 (m, 2H), 5.02-4.96 (m, 4H), 4.02 (q, J = 7.2 Hz, 2H), 3.93 (s, 3H), 3.10 (s, 3H), 2.83 (s, 3H), 0.82 (t, J = 7.2 Hz, 3H). | m/z 777 (M + 3H)⁺ (ES⁺) |

Entries marked * are parent active compounds of prodrugs of the invention

Biological Testing

There is provided below a summary of the biological assays performed with all the compound of the invention, and further assays performed with the compounds UL1-005 and U L1-012, UL1-027 and UL1-114 as comparators.

A. Primary in Vitro Assays: Inhibition of the Haemolytic Activity of Pneumolysin

Rationale

The basis of this assay is that when pneumolysin is added to red blood cells, it induces their lysis and leads to the release of haemoglobin. In the presence of an inhibitory compound, pneumolysin-induced lysis is abolished, the red blood cells pellet at the bottom of the microtitre plate well and the supernatant is clear. However, if the compound is not inhibitory, the red blood cells are lysed and haemoglobin is released into the supernatant.

Experimental Procedure

Test compound solutions (typically at 5 mM in DMSO) were diluted 1:1 in 100% DMSO. The compounds were then two-fold serially diluted in 100% DMSO across 11 wells of 96-well round-bottomed microtitre plate. PBS was then added to all the wells to achieve a 1:10 dilution of the compound in PBS. Pneumolysin was then added at a concentration equal to its LD100. Plates were then incubated at 37° C. for 30-40 min. After the incubation period, an equal volume of 4% (v/v) sheep erythrocyte suspension was added to each well and the plates incubated again at 37° C., for at least 30 min. Controls with only erythrocytes in PBS (control for no lysis) or erythrocytes plus pneumolysin (control for lysis) were prepared following the same procedure. Following the incubation with the erythrocytes, the Absorbance at 595 nm of each well was measured and the data used to determine the IC₅₀ for each test compound. The IC₅₀ values were determined using non-linear regression curve fitting. For that, the Log of the concentrations of the test compound was plotted against the percentage inhibition, estimated from the A₅₉₅ values, followed by fitting a Hill Slope to the data.

Results

This assay is principally relevant for the determination of the inhibitory activity of the parent active compounds UL1-005, UL1-012 and UL1-027. Generally, in the case of the prodrug, the inhibitory activity is expected to be absent in vitro, as the prodrug requires the presence of plasma enzymes to hydrolyse the prodrug moiety and allow the formation of the parent active compound. However, in our primary in vitro assay, blood is a component of the assay and is used to assess the inhibition of haemolysis induced by pneumolysin. Therefore, we observe inhibitory activity in the presence of the prodrugs of this invention, due to the enzymatic cleavage of the prodrug moieties, occurring during the 40 min incubation in blood, which leads to the release of the parent active compounds. In summary, this assay demonstrates the in vitro activity of the parent active compounds UL1-005, UL1-012 and UL1-027, and indicates that the prodrugs UL1-114, UL6-002, UL6-004, UL6-006 and UL6-008 convert to the parent active compounds in the presence of blood. This conversion to the parent active compounds is further demonstrated in Section F. IC₅₀ values are shown in Table 2:

TABLE 2

| Example | Prodrug/Active | IC₅₀ (μM) |
|---|---|---|
| UL1-005 | Active | 0.2 |
| UL1-012 | Active | 0.2 |
| UL1-027 | Active | 0.2 |
| UL1-114 | Prodrug | 19.5 |
| UL6-002 | Prodrug | 2.0 |
| UL6-004 | Prodrug | 1.6 |
| UL6-006 | Prodrug | 0.3 |
| UL6-008 | Prodrug | 1.9 |

B. Secondary in Vitro Assay: Inhibition of Pneumolysin-Induced Lactate Dehydrogenase Release

Rationale

Pneumolysin induces the release of lactate dehydrogenase (LDH) from human monocytes and lung epithelial cells: a phenomenon that is indicative of plasma membrane damage or rupture [Infect. Immun. (2002) 70 1017-1022]. The LDH assay was applied to demonstrate the ability of the disclosed compounds to inhibit the cytotoxic effect of pneumolysin on human lung epithelial cells in culture. The use of this assay can provide two main pieces of information on (1) Activity, to demonstrate the inhibition of LDH release from cells exposed to pneumolysin in the presence of inhibitory compounds versus the LDH release from cells exposed to pneumolysin alone, (2) Compound toxicity, the assay format was designed so it allows, in the control wells, the testing of the LDH release from cells exposed to the compound only.

Experimental Procedure

Human lung epithelial cells (A549) were seeded in flat-bottomed 96-well tissue culture plates and grown in RPMI 1640 medium supplemented with Glutamine, at 37° C., 5% CO₂, for 24 h.

Before use, the cells were washed with PBS. Test compound dilutions were incubated with pneumolysin as described in Section A, then transferred to wells containing the human lung epithelial cells and the plates were incubated at 37° C., 5% CO₂, for 30 min. The following controls were included on the plate (1) Negative controls, called low control (PBS only) to measure the natural release of LDH from the cells in culture, (2) positive controls (1% (v/v) Triton-X in PBS) to measure the maximum release of LDH from the cells (3) Pneumolysin solution only to measure pneumolysin-induced LDH release, (4) Test compound solution to assess the toxicity of the compound alone. After incubation, the supernatant was transferred to the wells of round-bottomed 96-well microtitre plates containing a double volume of lactate dehydrogenase assay mixture (TOX7, Sigma) prepared according to manufacturer's instructions. Incubation in a light-proof chamber at RT for 5-10 min was followed by the addition of 1N HCl to all wells. Absorbance at 490 nm and 655 nm was then measured. The percentage of LDH release induced by pneumolysin in the presence and absence of test compounds was plotted against the Log of the concentration of the compound and the IC₅₀ was determined, as described above in the inhibition of haemolysis assay, Section A.

Results

UL1-005, the parent active compound of the prodrugs UL6-005 and UL6-006, was tested in the LDH assay in triplicate over a range of concentrations from 62.5 μM to 0.49 μM. The results obtained are shown in FIG. 1.

In FIG. 1: (1) The horizontal dotted line at 100%, PLY control (--), indicates the maximum release of LDH from the cells under the effect of pneumolysin, as opposed to the horizontal solid line at 0% (low control), which corresponds to the supernatant of cells exposed to the assay buffer alone that shows the natural LDH release under the assay conditions. (2) The grey solid line (-▴-) shows that the LDH release from cells exposed to pneumolysin was significantly reduced in the presence of UL1-005, in a dose response manner, when compared to the PLY control. This demonstrates that UL1-005 prevents pneumolysin from damaging the human lung epithelial cells in culture, with an IC₅₀<0.49 μM. (3) The solid black line (-×-) shows that UL1-005 does not exhibit cytotoxicity at the concentrations tested, up to approximately 150 times the therapeutic IC₅₀ value.

Conclusion

UL1-005 inhibits the damaging activity of pneumolysin on human lung epithelial cells in culture. UL1-005 did not exhibit cytotoxic effects on the human lung epithelial cells at 150 times the therapeutic IC₅₀ value.

C. Ex Vivo Assay: Inhibition of the Effect of Pneumolysin on the Ciliary Function of Cultered Ependymal Cells

Rationale

The ependymal ciliated cells line the cerebral ventricles of the brain and the central canal of the spinal cord and are covered with cilia responsible for the circulation of the cerebrospinal fluid (CSF) around the central nervous system. This layer acts as a selective brain barrier to and from the cerebrospinal fluid and plays a role in controlling the CSF volume. To study if the inhibitors prevent the damage caused by pneumolysin on the ependymal layer, a rat ex vivo model of meningitis was used. This model is based on culturing and differentiating ciliated ependymal cells from neonate rat brains, which recreate the in vivo situation, where cells lining the brain ventricles, are exposed to S. pneumoniae and its toxic products.

The use of the ex vivo model of meningitis constitutes a powerful means to predict the ability of a compound to prevent pneumolysin from causing damage in vivo.

Experimental Procedure

Ependymal cell cultures were prepared by the method previously described [Microb. Pathog. (1999) 27 303-309]. Tissue culture trays were coated with bovine fibronectin and incubated at 37° C. in 5% (v/v) CO₂ for 2 h before use. The growth medium was minimum essential medium (MEM) with added penicillin (100 IU/mL), streptomycin (100 μg/mL), fungizone (2.5 μg/mL), BSA (5 μg/mL), insulin (5 μg/ml), transferrin (10 μg/mL) and selenium (5 μg/mL). Neo-natal (0-1 day old) rats were killed by cervical dislocation, and their brains were removed. The cerebellum was removed along with edge regions of the left and right cortical hemispheres and the frontal cortex. The remaining brain areas were mechanically dissociated in 4 mL of growth medium. The dissociated tissue from one or two brains was added to the wells of the tissue culture trays (500 μl/well), each containing 2.5 mL of growth medium. The cells then were incubated at 37° C. in 5% (v/v) CO₂. The medium was replaced after three days and thereafter the ependymal cells were fed every two days with 2 mL of fresh growth medium supplemented with thrombin.

After approximately two weeks, the cells were fully ciliated and ready for experiments. To perform the experiments, the growth medium was replaced with 1 mL of medium MEM containing 25 mM HEPES, pH 7.4. The tissue culture trays were placed inside a thermostatically controlled incubation chamber surrounding the stage of an inverted light microscope. The cell cultures were allowed to equilibrate until the temperature of the assay medium was 37° C. At this point, recombinant purified pneumolysin, with and without test compound, pre-incubated in 1 ml of medium MEM at 37° C. for 40 min, was added to the wells containing the ciliated cells. To the control cells, 1 mL of MEM medium was added. Beating cilia were recorded before and after exposure over 30 min, with a digital high-speed video camera at a rate of 500 frames/s. The recorded video sequences were played back at reduced frame rates and the ciliary beat frequency (CBF) was determined by the following equation:

$$CBF ( Hz ) = \frac{500 frames / s}{( frames elapsed for 5 ciliary beat cycles )} \times 5 ( conversion per beat cycle ) .$$

Results

The parameter measured was the ciliary beating frequency (CBF). Pneumolysin added to 40 ciliated cells in culture induces a severe or total loss of ciliary beating. UL1-005, the parent active compound of prodrug U L5-001, inhibited this damaging effect induced by pneumolysin on the ciliary function of ependymal cells in culture (FIG. 2).

In FIG. 2: Each time point represents the normalised mean±SD of ciliary beating frequency (CBF) measurements of ten individual cilia from each well, in three independent experiments. (1) Control 1, assay medium only: the symbol (-|-) represents measurements of the CBF in the assay medium which was used as a reference for the normal cilia beating. No damaging effect on the CBF was seen throughout the recording. (2) Control 2, pneumolysin only: The symbol (--) represents measurements of the CBF in the wells where pneumolysin was added. A substantial drop in the CBF to 0% of the original frequency was observed within 5 min. of exposure to the cytotoxin. (3) Treatment with UL1-005: The symbol (-▾-) represents the measurements of the CBF in the presence of pneumolysin and UL1-005 (1.56 μM). No significant loss of the CBF was seen, showing that UL1-005 inhibits pneumolysin-induced damage on the ciliary beating frequency of the brain ependymal cells. There was no statistical difference between the CBF of Control 1 (medium only) and the CBF in the presence of the treatment (-▾-) indicating that the inhibition of the damaging effect of pneumolysin by UL1-005 was achieved to an extent comparable to the control medium alone.

Conclusion

UL1-005 inhibits the damaging effect that pneumolysin induces on ependymal ciliated cells in culture. This predicts that when the prodrugs UL6-005 and UL6-006 are converted in vivo to the parent active compound UL1-005, the latter will prevent pneumolysin from causing damage in vivo.

D. Solubility and Chemical Stability Testing for the Determination of a Formulation Suitable for Intravenous Administration

Rationale

Parenteral delivery is one preferred route of administration of compounds of the invention. Therefore, aqueous solubility and chemical stability in the formulation are important parameters for the pharmaceutical utility of the compounds of the invention. The prodrugs of the invention were designed to improve the solubility and the chemical stability in solution of the parent active compounds and were optimised to achieve a readily soluble and stable formulation that could be reconstituted at the bed side and at high concentrations in safe saline solutions, at a pH compatible with intravenous administration. Once the formulation is administered intravenously, the prodrugs will be enzymatically cleaved in the circulation to release their parent active compound. The cleavage of the prodrugs to their corresponding parent active compound in the presence of blood is demonstrated in Section F.

Examples on the improvement of the solubility and chemical stability in formulations are shown below for prodrugs UL6-006 and UL6-008 and their corresponding parent active compounds UL1-005 and UL1-012.

Experimental Procedure

Solubility Testing

Solubility studies were performed by charging a vial with 5-10 mg of compound followed by the 40 addition of PBS solution or 0.9% saline to achieve a concentration of 100 mg/ml. If solubility was not observed the solution was diluted to concentrations of 50 mg/ml, 25 mg/ml and 4 mg/ml consecutively until complete solubility was observed.

Chemical Stability Assessment

Stability studies were performed by dissolving 1-2 mg of compound in DMSO (1 ml) followed by addition of 0.4 ml of the resulting solution to stirred PBS (9.6 ml) at 37.5° C. A sample (˜0.5 ml) was immediately taken for HPLC analysis. Further samples were then taken for analysis at various time-points thereafter. Half-lives were determined from the decrease in concentration of compound with respect to time.

Results

The formulations obtained with prodrugs UL6-006 and UL6-008 and their corresponding parent active compounds are shown in Table 3. Higher solubility and enhanced chemical stability was obtained with prodrugs UL6-006 and UL6-008 in comparison to their corresponding parent active compounds UL1-005 and UL1-012. Therefore the prodrugs are selected for their pharmaceutical utility by offering safe formulations that are readily soluble, with enhanced chemical stability at high concentrations and at a pH compatible with intravenous administration.

TABLE 3
Properties of the formulations of compounds of the invention

| | | Solubility in 0.9% (w/v) | Chemical stability | |
|---|---|---|---|---|
| | | saline, pH 6.5 or PBS* | at pH 6.2-7.2 | |
| Example | | pH 7.2 | (t_{1/2}) | |
| UL1-005 | Active | Not soluble | <30 | minutes |
| UL1-012 | Active | Not soluble | 61 | minutes |
| UL6-006 | Prodrug of | Soluble at 50 mg/mL in | 29.5 | hours |
| | UL1-005 | PBS, pH 5-6 | | |
| UL6-008 | Prodrug of | Soluble at 100 mg/mL in | 6 | days |
| | UL1-012 | PBS, pH 6 | | |

*Phosphate Buffered Saline

E. In vivo Efficacy Assay Using a Mouse Pneumonia Model

Rationale

This model has been well established in the laboratory of the inventors and has become adapted by other research groups working in this field. Using this model, pneumolysin was shown to be essential for the pathogenesis of S. pneumoniae and for its survival in vivo. With this disease model, mice infected with a strain of S. pneumoniae mutant deficient in pneumolysin (PLN-A), exhibited (1) a significant increase in the survival, (2) significant delay and attenuation of the signs of the disease and (3) substantial decrease in the pulmonary inflammation and less bacteraemia (infiltration of the bacteria from the lungs to the circulation). Therefore, this in vivo disease model constitutes a powerful tool to study the disease progression of mice infected with wild-type S. pneumoniae and treated with pneumolysin inhibitors. Survival was used as an endpoint parameter for the study.

Experimental Procedures: Infection, Treatment and Disease Signs Scoring

Outbred MF1 female mice, 8 weeks old or more and weighing 25-30 g may be used. The animals are maintained under controlled conditions of temperature, humidity and day length. They have free access to tap water and pelleted food. The in vivo experiments are performed using two control groups: Control 1 (infected and not treated), Control 2 (not infected and treated) and one Treatment group (infected and treated). Mice in control group 1 and in the treatment group are infected intranasally with Streptococcus pneumoniae strain D39 (procedure described below). After completing the infections, the viable count of the given dose is determined (as described below). Subsequently, every six hours, animals in the treatment group and in the control group 2, receive the test compound intravenously, while excipient alone is administered to control group 1. The progress of the signs of disease (Table 4) is assessed 5 every 6 h based on the scheme of Morton and Griffiths [Veterinary Record. (1985) 111, 431-436]. Animals are killed if they became 2+ lethargic and the time was recorded. The survival rates of control and test groups are compared with a log-rank test.

TABLE 4
Scoring scheme of the disease signs

| Sign | Description |
|---|---|
| Normal | Healthy appearance. Highly active. |
| 1+/2+ Hunched | Slight (1+) or pronounced (2+) convex curvature of the |
| | upper spine. |
| 1+/2+ Starey coat | Slight (1+) or pronounced (2+) piloerection of the coat. |
| (Piloerection) | |
| 1+/2+ Lethargic | Pronounced hunching and piloerection accompanied by |
| | a considerable (1+) or severe (2+) reduction of |
| | activity. |

The experimental design is shown in FIG. 3. The procedures used for infection with S. pneumoniae, the delivery of the treatment and for the determination of the bacterial viable counts, mentioned above, are detailed as follows:

Intranasal Instillation of Infection

Mice are lightly anaesthetised with 2.5% (v/v) isoflurane over 1.6-1.8 L O₂/min. The confirmation of effective anaesthesia is made by observation of no pedal reflex. A mouse is held by the scruff of the neck in a vertical position with its nose upward. The infectious dose is then administered in sterile PBS, given drop by drop into the nostrils, allowing the animal to inhale it in between drops. Once the dose is given, the mouse is returned to its cage, placed on its back to recover from the effects of anaesthetic.

Intravenous Administration of Treatment

Mice are placed inside an incubator at 37° C., for 10 min, to dilate their veins. Each mouse is then individually placed inside a restrainer, leaving the tail of the animal exposed. The tail is disinfected with antimicrobial wipes. The treatment with the compound of the invention is administered intravenously every 6 h using a 0.5 ml insulin syringe inserted carefully into one of the tail lateral veins. Doses are prepared freshly and administered intravenously to the animals.

Determination of Viable Count of the Infectious Dose

Viable counting is performed by the method of Miles and Misra [J. Hyg. (1938) 38 732-749). 20 μL of the sample are serially diluted in 180 μL PBS in round-bottomed 96-wells microtitre plates, up to a dilution of 10⁶. Blood agar plates are divided into six sectors and 60 μL of each dilution plated onto an individual sector. The plates are incubated in CO₂ gas jars overnight at 37° C. The following day, colonies are counted in the sector where 30-300 colonies are visible. The concentration of colony forming units (CFU) per millilitre is determined by using the following equation:

$$CFU per ml = \frac{Number of colonies in sector}{60 μl} \times Dilution \times 1000 ( conversion factor ) .$$

F. Conversion of Prodrug Derivatives to Active Inhibitors in Mouse, Rat or Human Plasma

Rationale

To demonstrate that the prodrug derivatives are converted to the parent active compound in the presence of plasma enzymes, a prodrug derivative was incubated with mouse, rat or human plasma at 37° C. at 5 time points over a 2 h period. The samples were then analysed by LC-MS/MS to obtain the amount of parent active compound appearing and prodrug derivative remaining over time.

Experimental Procedure

Prodrug derivatives were assessed in the mouse, rat or human plasma stability assay at a concentration of 10 μM. Test compounds were diluted in DMSO to a final stock concentration of 10 mM. For the purpose of the assay, the stocks prepared were further diluted in DMSO to a concentration of 400 μM and 5 μL were added to 195 μL of mouse, rat or human plasma (pH 7.4) and then incubated at 37° C. The final concentration of DMSO in the plate was 2.5% (v/v). Reactions were terminated at 0, 15, 30, 60 and 120 min after incubation by adding 400 μl of acetonitrile containing 0.55 μM metoprolol and 1% (v/v) formic acid. The plate was then centrifuged at 3000 rpm, for 45 min, at 4° C. 80 μL of supernatant were transferred into a conical bottom 96 well glass coated plate. 40 μL of water were added prior to analysis for prodrug derivative and active species by LC-MS/MS. This assay was performed by a contract research organisation, Cyprotex Discovery Limited, UK, at the request of the inventors at Leicester.

Results

The quantification of the prodrug derivative remaining and the parent active compound appearing was performed as follows:

(1) The parent active compound was quantified using a 6 point calibration curve prepared in deactivated mouse, rat or human plasma. (2) The percentage of prodrug compound remaining at each time point relative to 0 min sample was calculated from LC-MS/MS peak area ratios (compound peak area/internal standard peak area). This percentage was then used to determine the concentration of the prodrug compound at each time point in reference to the starting concentration (10 μM) at time 0 min.

A summary of the conversion of the prodrugs UL6-002, UL6-004, UL6-006 and UL6-008 to the respective parent active compounds UL1-005, UL1-012 and UL1-027 is shown in Table 5.

Conclusion

The results presented in Table 5 clearly indicate the therapeutic benefits of the prodrugs of the invention, which is demonstrated by their conversion in plasma into the parent active compounds. In addition, the physicochemical properties of the prodrugs U L6-002, UL6-006 and UL6-008 are favourable for the preparation of formulations suitable for parenteral delivery.

TABLE 5

| | | [ ] | [ ] | [ ] | [ ] | [ ] |
|---|---|---|---|---|---|---|
| Prodrug | Prodrug/ | (μM) | (μM) | (μM) | (μM) | (μM) |
| ID | Active* | t_{0 min.} | t_{15 min.} | t_{30 min.} | t_{60 min.} | t_{120 min.} |
| UL6- | Prodrug | 10.00 | 0.00 | 0.00 | 0.02 | 0.00 |
| 002 | (Mouse) | | | | | |
| | (UL6-002) | | | | | |
| | Active | 0.00 | 4.69 | 4.78 | 4.61 | 4.63 |
| | (Mouse) | | | | | |
| | (UL1-027) | | | | | |
| UL6- | Prodrug | 10.00 | 10.00 | 9.88 | 9.80 | 9.63 |
| 004 | (Mouse) | | | | | |
| | (UL6-004) | | | | | |
| | Active | 0.00 | 0.11 | 0.38 | 0.84 | 1.40 |
| | (Mouse) | | | | | |
| | (UL1-027) | | | | | |
| UL6- | Prodrug | 10.00 | 2.05 | 0.79 | 0.10 | 0.04 |
| 006 | (Mouse) | | | | | |
| | (UL6-006) | | | | | |
| | Active | 0.05 | 4.71 | 9.81 | 12.50 | 9.17 |
| | (Mouse) | | | | | |
| | (UL1-005) | | | | | |
| | Prodrug | 10.00 | 8.90 | 1.17 | 0.21 | 0.07 |
| | (Rat) | | | | | |
| | (UL6-006) | | | | | |
| | Active | 0.19 | 4.98 | 7.69 | 11.27 | 10.85 |
| | (Rat) | | | | | |
| | (UL1-005) | | | | | |
| | Prodrug | 10.00 | 2.15 | 0.05 | 0.01 | — |
| | (Human) | | | | | |
| | (UL6-006) | | | | | |
| | Active | 0.09 | 10.02 | 11.34 | 11.51 | 10.26 |
| | (Human) | | | | | |
| | (UL1-005) | | | | | |
| UL6- | Prodrug | 10.00 | 4.53 | 1.70 | 0.47 | 0.09 |
| 008 | (Mouse) | | | | | |
| | (UL6-008) | | | | | |
| | Active | 0.05 | 6.37 | 10.53 | 12.25 | 12.82 |
| | (Mouse) | | | | | |
| | (UL1-012) | | | | | |
| | Prodrug | 10.00 | 2.90 | 1.00 | 0.21 | 0.07 |
| | (Rat) | | | | | |
| | (UL6-008) | | | | | |
| | Active | 0.04 | 3.38 | 6.52 | 9.67 | 8.65 |
| | (Rat) | | | | | |
| | (UL1-012) | | | | | |
| | Prodrug | 10.00 | 4.89 | 5.10 | 6.06 | 4.52 |
| | (Human) | | | | | |
| | (UL6-008) | | | | | |
| | Active | 0.08 | 0.28 | 0.55 | 1.40 | 2.70 |
| | (Human) | | | | | |
| | (UL1-012) | | | | | |

Conclusion

The results presented in Table 5 clearly indicate the therapeutic benefits of the prodrugs of the invention, which is demonstrated by their conversion in plasma into the parent active compound. Besides the demonstration of its therapeutic benefits, the physicochemical properties of the compounds of the invention are favourable for the preparation of formulations and are particularly suitable for parenteral delivery.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All patents and patent applications referred to herein are incorporated by reference in their entirety.

1. A compound selected from: 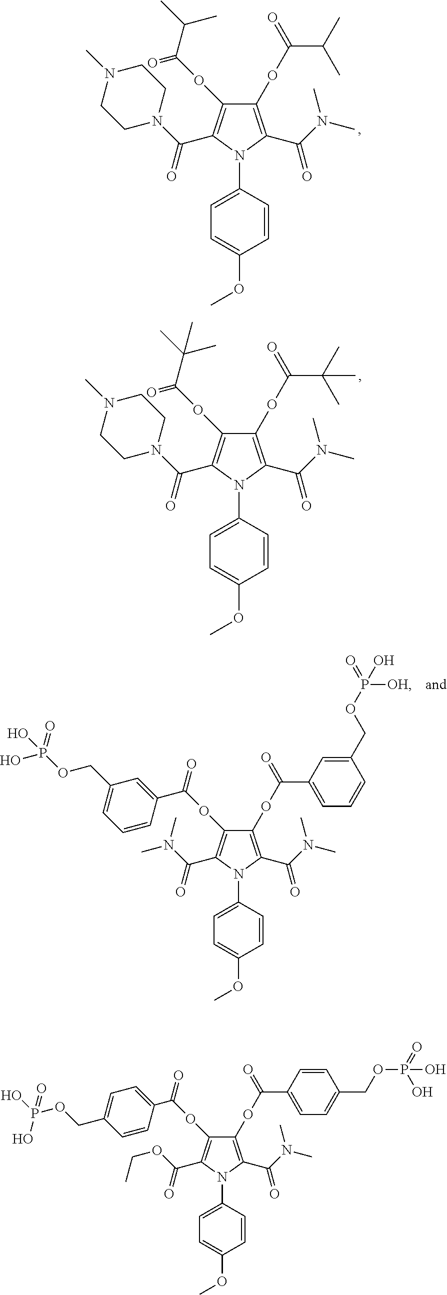
and pharmaceutically acceptable salts and solvates thereof.

2. A compound according to claim 1 selected from:
2-(Dimethylcarbamoyl)-1-(4-methoxyphenyl)-5-(4-methylpiperazine-1-carbonyl)-1H-pyrrole-3,4-diyl bis(2-methylpropanoate),
2-(D imethyl carbamo y1)-1-(4-methoxyphenyl)-5-(4-methylpiperazine-1-carbonyl)-1H-pyrrole-3,4-diyl bis(2-methylpropanoate) hydrochloride,
2-(Dimethylcarbamoyl)-1-(4-methoxyphenyl)-5-(4-methylpiperazine-1-carbonyl)-1H-pyrrole-3,4-diyl bis(2,2-dimethylpropanoate),
2-(Dimethylcarbamoyl)-1-(4-methoxyphenyl)-5-(4-methylpiperazine-1-carbonyl)-1H-pyrrole-3,4-diyl bis(2,2-dimethylpropanoate) hydrochloride,
2,5-Bis(dimethylcarbamoyl)-1-(4-methoxyphenyl)-1H-pyrrole-3,4-diyl bis(3-((phosphonooxy)methyl)benzoate),
Sodium ((((2,5-bis(dimethylcarbamoyl)-1-(4-methoxyphenyl)-1H-pyrrole-3,4-diyl)bis(oxy))bis(carbonyl))bis(3,1-phenylene))bis(methylene) bis(hydrogenphosphate),
2-(Dimethylcarbamoyl)-5-(ethoxycarbonyl)-1-(4-methoxyphenyl)-1H-pyrrole-3,4-diyl bis(4-((phosphonooxy)methyl)benzoate), and
Sodium ((((2-(dimethylcarbamoyl)-5-(ethoxycarbonyl)-1-(4-methoxyphenyl)-1H-pyrrole-3,4-diyl)bis(oxy))bis(carbonyl))bis(4,1-phenylene))bis(methylene) bis(hydrogenphosphate).

3. A pharmaceutical composition comprising a compound according to claim 1, optionally in combination with one or more pharmaceutically acceptable diluents or carriers.

4. A pharmaceutical composition according to claim 3 comprising one or more other therapeutically active ingredients.

5. (canceled)

6. A compound according to claim 1 for use in combination with one or more other therapeutically active ingredients.

7. A method of treating bacterial infections caused by bacteria producing pore-forming toxins, such as cholesterol dependent cytolysins, in a subject, the method comprising administering to the subject a therapeutically effective amount of a compound according to claim 1.

8. The method according to claim 7 wherein the bacterial infection is caused by Streptococcus spp. (e.g. Streptococcus pneumoniae, Group A Streptococci or Streptococcus suis), Clostridium spp. (e.g. Clostridium perfringens), Listeria spp. (e.g. Listeria monocytogenes) or Bacillus spp. (e.g. Bacillus anthracis).

9. The method according to claim 8 wherein the bacterial infection is caused by Streptococcus pneumoniae.

10. The method according to claim 9 for the treatment of pneumococcal pneumonia, pneumococcal meningitis, pneumococcal septicaemia/bacteraemia, pneumococcal keratitis or pneumococcal otitis media.

11. The method according to claim 7 for the treatment of conditions selected from gas gangrene, gastrointestinal anthrax, inhalational anthrax, porcine meningitis, encephalitis, septicaemia/bacteraemia and pneumonia which are caused by bacteria other than pneumococcus.

12. The method according to claim 5 wherein the compound is administered in combination with one or more additional therapeutically active ingredients (e.g. one or more antimicrobial or immunomodulatory agents).

13. (canceled)

14. A process for preparing a compound according to claim 1 which comprises reacting a compound of formula (I): 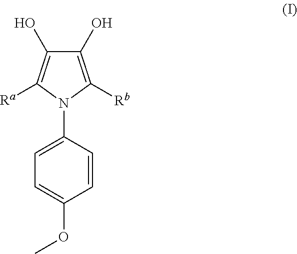
wherein R^{a} and R^{b} correspond to the 2- and 5-position substituents in the compounds of claim 1, with:
a) 3-((phosphonooxy)methyl) benzoic acid), or a protected derivative thereof, e.g. a di-tert-butyl protected derivative thereof, followed if required by deprotection; or
b) a compound of formula LG-C(O)—R^{c}, where LG is a leaving group, e.g. chloro, and R^{c} is —C(CH³)₃ or —CH(CH₃)₂;
and optionally forming a salt or solvate thereof.
